SEQUENCE LISTING (14 sequences)

SEQ ID NO: 1 - protein, 186 aa, Trichoderma reesei
APLPAPPGIPSEDTARTQLAGLTVAVVGSGTGYSRDLFPTWDAISGNCNAREYVLKRDGEGVQVNNACEAQSGSWISPYDNASFTNASSLDIDHMVPLKNAWISGASTWTTAQREALANDVSRPQLWAVSASSNRSKGDRSPDQWKPPLTSFYCTYAKSWIDVKSYYKLTITSAEKTALSSMLDTC

SEQ ID NO: 2 - protein, 219 aa, Tolypocladium inflatum
APFPEPAPSLEAESWTDDAIQPGIPSASTARTLLAGLQVAASGSGTGYSREKFPHWATISGTCNARYDPSRGTVSRQLTLACRSEFVLKRDGENVVVNSACTAESGTWKSPYDGASWTQASDIDIDHMVPLKNAWISGASSWTTDQRKGFANDITRPQLWAVTDNVNQAKSDSSPDRWKPPLTSFYCTYAKSWVQVKSYYKLTVTDAEKGALSGMLDSC

SEQ ID NO: 3 - protein, 190 aa, Blastomyces gilchristii
STIPLHKRDPPNIPSPPEASRLLELMVVSPQGPQTGYSRTKFPHWITINGPCNTRETVLKRDGMNVTVGKNCSPKSGSWYSTYDGVTLTSSSNVDIDHVVPLSDAWKSGASGWTRERRRDFANDMENPQLISVQGKVNKEKGDKGPDKWKPPLPTYHCTYACMWVKVKSAYNLTITDPEKQALMDMLGTC

SEQ ID NO: 4 - protein, 188 aa, Gelasinospora tetrasperma
APAPMPTPPGIPTAANAKTQLASLTVKAYVDDGKYERDLFPTWITISGTCNTREYVLKRDGTNVVTDSACTATSGTWASPYDGGVWTAASDVDIDHMVPLKNAWVSGANAWTTSKRQQFANDINSPQLWAVTDNVNQAKSDQSPDAWKPPLTSFYCTYAKAWVQVKYTWALTITSAEKSALTSMLNTC

SEQ ID NO: 5 - protein, 292 aa, Streptococcus dysgalactiae
KTVSVNQTYGEYKDYYTVIGESNIDQSAFPKIYKTTERVYKGQGTSEKRVTVSDVVYNPLDGYKRSTGAYGVVTKDMIDMSKGYREKWETNPEPSGWFRFYNRADNEEISEKEYDSRRTKSYKVTNNVPVVLTTLKGKKYNSHLFVASHLFADSLGGKSIRKNAITGTQMQNVGTRKGGMQYIEKKVLSHITKNPDVYVFYSAIPEYQGAELLARSVLVSALSSDGVINETVRVFNTADGFNINYEKGGLLTESPVSEIDNIEDSTTDEIENSVDDSEEIVYNDTTTEEEEN

SEQ ID NO: 6 - protein, 209 aa, Thermobifida cellulosilytica
LDDLLAELEEIEADVPATAESQAPEGDADAVEARQRLGEIPIAEPAPRGDYDRDEFGSGWIDTDGNGCSTRNDILARDLENVQLASDGCKVLSGTLHDPYTGATIEFTSEKPQAVQIDHVVPLSLAWRMGASRWDRDTRVEFANDPGNLLASDGPANREKSDSGPGEWQPYEGFQCTYAVLYIDVLYRYDLPVSEQDYEGLSAMLDTCA

SEQ ID NO: 7 - protein, 267 aa, Rasamsonia composticola
WGNLGHETIAYIAQNFVASSTASYCQDVLGDTSSSYLASVATWADSYRYTSAGRWSEPLHFIDAHDDPPTSCGVDYERDCGDKGCVVSAINNYTTQLLDKSTSSSEALDAMKFIIHFVGDIHQPLHDENLDVGGNDIDVTFNGTKTNLHHIWDTNMLEADAGGSSLSTAKSFAATLTTRLQSGEYASQKASWISGMNISDPVATAMGWASDANGYVCSTVLKPGLSYIKDNDLSGKYYADCKPVFEELLARAGYRLAAWLDLIAKQQ

SEQ ID NO: 8 - protein, 190 aa, Chaetosartorya cremea
IPVPVALPTPPGIPSAATAESELAALTVAAQGSSSGYSRDLFPHWITQSGTCNTREVVLARDGSNIVRDSSCYPTSGSWYSPYDGATWTQASDVDIDHVVPLSNAWKSGASKWTTPQRQAFANDLTNPQLMAVTDNVNQAKGDSGPEDWKPPLTSYYCTYAKMWVRVKYVYDLTITSAEKSALVSMLDRC

SEQ ID NO: 9 - skipped

SEQ ID NO: 10 - protein, 182 aa, Aspergillus bisporus
TPPGIPSTSSAKSQLAALTVRSAGSSSGYSRDLFPHWINQGNNCNTREVVLARDGSNVQTGSDCAPTSGSWYSPYDGATWYQASDVDIDHVVPLSNAWKSGASSWTTSRRQAFANDLSNPQLIAVTDNVNQSKGDKGPEEWKPPRTAYHCTYAKMWVRVKYVYGLTVTSSEKSALSSMLNTC

SEQ ID NO: 11 - protein, 174 aa, Aspergillus clavatus
LPTPPGIPSAATAESELASLTVAPQGSEDGYSRELFPHWINQGNHCNTRDMVLARDGQDVVQDDDCYPTSGTWYSPYDGTTWTEAKKLDIDHFVPLANAWRSGASEWSTAQRTAFANDLTHPQLVAVSEKANRSKGDKGPEEWKPPLASFHCTYARMWVSVKSTYALTITSEEK

SEQ ID NO: 12 - protein, 147 aa, Metarhizium acridum
VPIPEPTPSGIWTSPYDGLVFTEAHKLDIDHFVPLKNAWMVRKNQGPFPAGIVRIFAWLTCRLQSGASEWTDEQREAFANDLARPQLWAVSAHANRQKGDKGPDGWKPPLTSFFCTYAESWVEVKGYYNLTISESEKEALGSMLDDC

SEQ ID NO: 13 - protein, 182 aa, Aspergillus ochraceoroseus
TPPGIPSTSSAKSQLSSLTVAAQGSQSGYSRDLFPHWIDQGNSCNTREDVLIRDGTGVTTGSACKITAGSWYSPYDGATWTAASDVDIDHVVPLSNAWKSGASSWTTEERTAFANDLENPQLMAVTDNVNQAKGDKGPEDWQPPLSSYHCTYAKMWVRVKSVYDLTITSKEKSALQDMLATC

SEQ ID NO: 14 - protein, 182 aa, Bacillus cibi [sequence omitted]

(19) United States
(12) Patent Application Publication
(10) Pub. No.: US 2024/0384205 A1
(43) Pub. Date: Nov. 21, 2024

(54) LAUNDRY COMPOSITIONS FOR CLEANING

(71) Applicant: Danisco US Inc., Palo Alto, CA (US)

(72) Inventors: Jonathan Lassila, South San Francisco, CA (US); Zhenghong Zhang, Shanghai (CN); Kun Zhong, Shanghai (CN)

(21) Appl. No.: 18/687,521

(22) PCT Filed: Sep. 1, 2022
(86) PCT No.: PCT/US2022/042317

§ 371 Date: Feb. 28, 2024

(30) Foreign Application Priority Data
Sep. 3, 2021 (WO) PCT/CN2021/116480

(51) Int. Cl. C11D 3/386; C12N 9/22

(52) U.S. Cl. CPC C11D 3/38636; C12N 9/22; C12Y 302/01017

(57) ABSTRACT

Disclosed herein are compositions and methods for preventing, reducing, or removing biofilms.

### CROSS-REFERENCE OF RELATED APPLICATIONS

This application is a 371 of International Patent Application No. PCT/US22/042317 filed Sep. 1, 2022, which claims the benefit of PCT/CN21/116480, filed Sep. 3, 2021, each of which is hereby incorporated by reference in its entirety.

### REFERENCE TO THE SEQUENCE LISTING

The official copy of the sequence listing is submitted electronically via Patent Center as an XML formatted sequence listing with a file named 20240214 NB41734USPCT2 SequenceListing created on Feb. 14, 2024 and having a size of 14,601 bytes and is filed concurrently with the specification. The sequence listing contained in this XML formatted document is part of the specification and is herein incorporated by reference in its entirety.

### FIELD OF INVENTION

Described are deoxyribonucleases (DNases, nucleases, phosphodiesterases) for laundry or hard surface cleaning.

### BACKGROUND

Trends toward cold water washing and synthetic athletic wear are driving a need for detergents that eliminate bacteria and odor, while at the same time the industry is moving away from laundry powders where traditional oxygen bleach was feasible. Thus, a need exists for new approaches to remove malodor and microorganisms in laundry.

Formation of bacterial biofilms in washing machines and on laundry textiles contributes to the spread of harmful and malodorous bacteria (Bockmuhl 2017, Gattlen et al 2010). Biofilm formation increases the resistance of bacteria to removal and cleaning processes. This resistance is mediated by production of a biofilm extracellular matrix consisting of water, polysaccharides, proteins, nucleic acids, and lipids (Fleming et al., 2010; Limoli et al 2015; Kostakioti et al, 2013). Enzymes that degrade these extracellular matrix components can thus be used to reduce, inhibit, or remove bacterial biofilms (Kostakioti et al 2013, Fleming et al 2017)

Despite repeated exposure to surfactants, proteases, and amylases from typical laundry detergents, bacterial biofilms persist in washing machines and contribute to hygiene and odor problems (Bockmuhl 2017, Gattlen et al 2010). More effective solutions for removing biofilms in laundry are thus needed.

Nucleases have been found to disperse bacterial biofilms (e.g. Nguyen and Burrows (2014); Nijland et al (2010); Whitchurch et al (2002)) and have recently been of interest as potential laundry detergent additives (e.g. Morales-Garcia et al (2020), WO2015181287, WO2015155350, WO2016162556, WO2017162836, WO2017060475, WO2018184816, WO2018177936, WO2018177938, WO2018/185269, WO2018185285, WO2018177203, WO2018184817, WO2019084349, WO2019084350, WO2019081721, WO2018076800, WO2018185267, WO2018185280, and WO2018206553). However, laundry detergents pose special challenges for enzymes, with surfactants, chelants, proteases, and other components that can inactivate and denature enzymes. New types of nucleases that can withstand these difficult conditions while maintaining high performance are needed.

This invention disclosure describes new nucleases with improved stability and activity over previously described nucleases.

### SUMMARY

This invention describes nucleases which provide improvement in performance for laundry and home care applications, detergent compositions, in particular.

In an embodiment, the invention is a method for preventing, reducing or removing a biofilm comprising contacting the biofilm with a cleaning composition comprising a polypeptide having nuclease activity, wherein the polypeptide having the nuclease activity is a polypeptide having at least 80% sequence identity with an enzyme selected from the group consisting of SEQ ID NO: 1 to 13.

In another embodiment, the invention is a method for preventing, reducing or removing a biofilm from a textile or hard surface comprising: (i) contacting a textile or surface with a polypeptide having nuclease activity or a composition comprising a polypeptide having nuclease activity; and (ii) optionally, rinsing the textile or surface, wherein the polypeptide having the nuclease activity is a polypeptide having at least 80% sequence identity selected from the group consisting of SEQ ID NO: 1 to 13.

In another embodiment, the invention is a detergent composition.

In another embodiment, the invention is a detergent composition comprising: (i) a polypeptide having nuclease activity; (ii) a polypeptide having protease activity; (iii) at least one additional polypeptide, wherein the at least one additional polypeptide is an enzyme selected from: DNase, acyl transferases, alpha-amylases, beta-amylases, alpha-galactosidases, arabinosidases, aryl esterases, beta-galactosidases, carrageenases, catalases, cellobiohydrolases, cellulases, chondroitinases, cutinases, endo-beta-1,4-glucanases, endo-beta-mannanases, esterases, exo-mannanases, galactanases, glucoamylases, hemicellulases, hyaluronidases, keratinases, laccases, lactases, ligninases, lipases, lipoxygenases, lysozymes, mannanases, metalloproteases, nucleases, oxidases, oxidoreductases, pectate lyases, pectin acetyl esterases, pectinases, pentosanases, peroxidases, phenoloxidases, phosphatases, phospholipases, phytases, polygalacturonases, polyesterases, proteases, pullulanases, reductases, rhamnogalacturonases, beta-glucanases, tannases, transglutaminases, xylan acetyl-esterases, xylanases, xyloglucanases, xylosidases, and any combination or mixture thereof; and (iv) a surfactant, wherein the polypeptide having the nuclease activity is a polypeptide having at least 80% sequence identity selected from the group consisting of SEQ ID NO: 1 to 13.

In another embodiment, the invention is a method for reducing malodor associated with a textile or hard surface comprising: (i) contacting a textile or hard surface with a polypeptide having nuclease activity or a composition comprising a polypeptide having nuclease activity; and (ii) optionally, rinsing the textile or surface, wherein the polypeptide having the nuclease activity is a polypeptide having at least 80% sequence identity selected from a group consisting of SEQ ID NO: 1 to 13.

In another embodiment, the invention is an isolated polypeptide or active fragment thereof having nuclease activity, wherein the polypeptide comprises an amino acid sequence having at least 75%, 80%, 85%, 90%, 91%, 92%, 93%, 94%, 95%, 96%, 97%, 98%, 99%, or 100% sequence identity with an amino acid sequence selected from the group consisting of SEQ ID NO: 1 to 13.

### FIGURES

FIG. 1 provides a graphic representation of the results of one embodiment of the present disclosure providing data demonstrating dispersal of biofilm with 100 PPM enzyme. The figure shows the absorbance at 590 nm from crystal violet stain. The average value is plotted for all replicates, at least eight replicates per sample.

FIG. 2 provides a graphic representation of the results of one embodiment of the present disclosure providing data demonstrating dispersal of biofilm with 20 PPM enzyme. The figure shows the absorbance at 590 nm from crystal violet stain. The average value is plotted for all replicates, at least eight replicates per sample.

FIG. 3 provides a graphic representation of the results of one embodiment of the present disclosure providing data demonstrating dispersal of biofilm with 10 PPM, 50 PPM, and 250 PPM enzyme, as indicated. The figure shows the absorbance at 590 nm from crystal violet stain. The average value is plotted for all replicates, at least eight replicates per sample.

FIG. 4 provides a graphic representation of the results of one embodiment of the present disclosure providing data demonstrating a test of the combination of lysozyme SmaLys1 and nuclease CcrNuc1. The figures show the average values for all replicates (eight per sample). The single enzyme or combination was dosed at 5 ppm total enzyme and compared with SEQ ID No. 14 at 5 ppm. Treatment was performed at 26 C with shaking for 6 hr.

FIG. 5 provides a graphic representation of the results of one embodiment of the present disclosure providing data demonstrating a test of the combination of lysozyme SmaLys1 and nuclease CcrNuc1. The figures show the average values for all replicates (eight per sample). The single enzyme or combination was dosed at 10 ppm total enzyme and compared with SEQ ID NO. 14 at 10 ppm. Treatment was performed at 26 C with shaking for 6 hr.

FIG. 6 provides a graphic representation of the results of one embodiment of the present disclosure providing data demonstrating biofilm dispersal of CcrNuc1 in combination with SmaLys1. The figure shows the average biofilm signal (590 nm) for all replicates (eight per sample. The different shades refer to different total protein concentrations (50 ppm, 12.5 ppm, 3.1 ppm, 0.78 ppm, or 0.2 ppm). For example, black bars indicate 50 PPM of CcrNuc1 alone, 50 PPM of SEQ ID No. 14 alone, or 25 PPM SmaLys1+25 PPM CcrNuc1. Tide 1:1200 alone was used as negative control. SEQ ID NO. 14 dosed at 50 ppm was used for comparison.

### DESCRIPTION

The present disclosure provides compositions (e.g. enzyme and detergent compositions) and methods using such compositions for the prevention, reduction or removal of biofilms, for example, from an article, such as a hard surface or textile. The compositions generally employ the use of at least one polypeptide having nuclease activity or a composition comprising a polypeptide having nuclease activity. The compositions also optionally comprise additional components of a cleaning detergent, such as one or more surfactants.

### Definitions

Prior to describing embodiments of present compositions and methods, the following terms are defined.

Unless defined otherwise herein, all technical and scientific terms used herein have the same meaning as commonly understood by one of ordinary skill in the art to which this invention pertains. Although any methods and materials similar or equivalent to those described herein find use in the practice of the present invention, the preferred methods and materials are described herein. Accordingly, the terms defined immediately below are more fully described by reference to the specification as a whole. Also, as used herein, the singular terms “a,” “an,” and “the” include the plural reference unless the context clearly indicates otherwise. It is to be understood that this invention is not limited to the particular methodology, protocols, and reagents described, as these may vary, depending upon the context they are used by those of skill in the art.

It is intended that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The term “biofilm” refers to community of microorganisms embedded in an extracellular polymer matrix attached to a surface. The extracellular polymer matrix is a polymeric conglomeration generally composed of extracellular DNA, proteins, and polysaccharides. A biofilm may have one or more microorganisms and further includes water and may include other trapped particles. The microorganisms may be gram positive or gram-negative bacteria (aerobic or anaerobic); algae, protozoa, and/or yeast or filamentous fungi. In some embodiments the biofilm is living cells including one or more bacterial genera of Acinetobacter sp., Aeromicrobium sp., Brevundimonas sp., Microbacterium sp., Micrococcus luteus, Pseudomonas sp. (e.g. Pseudomonas fluorescens), Staphylococcus sp. (e.g. Staphylococcus epidermidis), and Stenotrophomonas sp., Streptomyces sp., Listeria sp., Streptococcus sp., and Escherichia sp.

As used herein, “surface” means any structure having sufficient mass to allow for attachment of biofilm. Hard surfaces include, but are not limited to metal, glass, ceramics, wood, minerals (rock, stone, marble, granite), aggregate materials such as concrete, plastics, composite materials, hard rubber materials, and gypsum. The hard materials may be finished with enamels and paints. Hard surfaces are found, for example in water treatment and storage equipment and tanks; dairy and food processing equipment and facilities; medical equipment and facilities, such as surgical instruments and permanent and temporary implants; industrial pharmaceutical equipment and plants. Soft surfaces are, for example, hair and all types of textiles. Porous surfaces also may be found in certain ceramics as well as in membranes that are used for filtration. Other surfaces include, but are not limited to, ship hulls and swimming pools. Other surfaces may be biological surfaces, such as skin, keratin or internal organs.

The term “fabric” refers to, for example, woven, knit, and non-woven material, as well as staple fibers and filaments that can be converted to, for example, yarns and woven, knit, and non-woven fabrics. The term encompasses material made from natural, as well as synthetic (e.g., manufactured) fibers.

The term “malodor” refers to foul or bad smelling fabrics or textiles.

The term “textile”, as used herein, refers to any textile material including yarns, yarn intermediates, fibers, non-woven materials, natural materials, synthetic materials, and any other textile material, fabrics made of these materials and products made from fabrics (e.g., garments and other articles). The textile or fabric may be in the form of knits, wovens, denims, non-wovens, felts, yarns, and toweling. The textile may be cellulose based such as natural cellulosics, including cotton, flax/linen, jute, ramie, sisal or coir or manmade cellulosics (e.g. originating from wood pulp) including viscose/rayon, cellulose acetate fibers (tricell), lyocell or blends thereof. The textile or fabric may also be non-cellulose based such as natural polyamides including wool, camel, cashmere, mohair, rabbit and silk or synthetic polymers such as nylon, aramid, polyester, acrylic, polypropylene and spandex/elastane, or blends thereof as well as blends of cellulose based and non-cellulose based fibers. Examples of blends are blends of cotton and/or rayon/viscose with one or more companion material such as wool, synthetic fiber (e.g. polyamide fiber, acrylic fiber, polyester fiber, polyvinyl chloride fiber, polyurethane fiber, polyurea fiber, aramid fiber), and/or cellulose-containing fiber (e.g. rayon/viscose, ramie, flax/linen, jute, cellulose acetate fiber, lyocell). Fabric may be conventional washable laundry, for example stained household laundry. When the term fabric or garment is used, it is intended to include the broader term textiles as well. In the context of the present application, the term “textile” is used interchangeably with fabric and cloth.

As used herein, the term “hard surface” refers to any article having a hard surface including floors, tables, walls, roofs etc. as well as surfaces of hard objects such as cars (car wash), ship hulls, dishes (dishware), medical instruments, pipes, reservoirs, or holding tanks. The term “hard surface” includes also the surfaces of flexible yet firm objects such as the insides of bendable tubing and supply lines or the surfaces of deformable holding tanks or vessels. The term “hard surface” includes also the surfaces in the interior of washing machines, such as the interior of laundry washing machines or dishwashing machines, this includes soap intake box, walls, windows, baskets, racks, nozzles, pumps, sump, filters, pipelines, tubes, joints, seals, gaskets, fittings, impellers, drums, drains, traps, coin traps inlet and outlets. The term hard surface does not encompass textile or fabric.

The term “laundering” includes both household laundering and industrial laundering and means the process of treating textiles with a solution containing a cleaning or detergent composition as provided herein. The laundering process can for example be carried out using e.g. a household or an industrial washing machine or can be carried out by hand.

The term “wash cycle” refers to a washing operation in which textiles are immersed in a wash liquor, mechanical action of some kind is applied to the textile to release stains or to facilitate flow of wash liquor in and out of the textile and finally the superfluous wash liquor is removed. After one or more wash cycles, the textile is generally rinsed and dried.

The term “wash liquor” is defined herein as the solution or mixture of water and detergent components optionally including polypeptides having nuclease activity.

### Polypeptides, Polynucleotides, and Expression Cassettes

In one embodiment, polypeptides are provided that have nuclease activity (e.g. Dnase activity) activity and at least 70% sequence identity to an amino acid sequence selected from the group consisting of SEQ ID NO: 1-13. The polypeptides having nuclease activity of the present disclosure include isolated, recombinant, substantially pure, or non-naturally occurring polypeptides. In some embodiments, the polypeptides are useful in cleaning applications and can be incorporated into cleaning compositions (e.g. detergent compositions) that are useful in methods of cleaning as provided herein.

In some embodiments, the polypeptides having nuclease activity provided herein include those having amino acid sequences having at least 70%, 75%, 80%, 85%, 90%, 91%, 92%, 93%, 94%, 95%, 96%, 97%, 98%, 99%, or 100% sequence identity with the amino acid sequence selected from the group consisting of SEQ ID NOs: 1-13.

In some embodiments, the nuclease polypeptides provided herein for use in the compositions and methods provided herein includes a polypeptide having an amino acid sequence having at least 50%, 55%, 60%, 65%, 70%, 75%, 80%, 85%, 90%, 91%, 92%, 93%, 94%, 95%, 96%, 97%, 98%, 99%, or 100% sequence identity with the amino acid sequence selected from the group consisting of SEQ ID NOs: 1-13.

Also provided is a variant nuclease polypeptide enzyme of SEQ ID Nos: 1-13, having nuclease activity, where the enzyme comprises an amino acid sequence which differs from the amino acid sequence of SEQ ID NOs: 1-13 by no more than 50, no more than 40, no more than 30, no more than 25, no more than 20, no more than 15, no more than 10, no more than 9, no more than 8, no more than 7, no more than 6, no more than 5, no more than 4, no more than 3, no more than 2, or no more than 1 amino acid residue(s), when aligned using alignment methods provided herein.

The variant enzyme polypeptides of the disclosure have enzymatic activities (e.g., nuclease activities) and thus are useful in a variety of cleaning applications, including but not limited to, methods for cleaning dishware items, tableware items, fabrics, textiles, and items having hard surfaces (e.g., the hard surface of a table, table top, wall, furniture item, floor, ceiling, etc.). Exemplary cleaning compositions comprising one or more polypeptides having nuclease activity of the disclosure are described infra. The enzymatic activity (e.g., nuclease activity) of an enzyme polypeptide of the invention can be determined readily using procedures well known to those of ordinary skill in the art. The Examples presented infra describe methods for evaluating the enzymatic activity and cleaning performance. The performance of polypeptide enzymes of the invention in reducing, preventing, and/or removing a biofilm can be readily determined using procedures well known in the art and/or by using procedures set forth in the Examples.

In some embodiments, the polypeptides of the present disclosure can have nuclease activity over a broad range of pH conditions. In some embodiments, the polypeptides have nuclease activity as demonstrated using the methods described in the examples. In some embodiments, the polypeptides have nuclease activity at a pH of from about 4.0 to about 12.0. In some embodiments, the polypeptides have nuclease activity at a pH of from about 6.0 to about 12.0. In some embodiments, the polypeptides have at least 50%, 60%, 70%, 80% or 90% of maximal nuclease activity at a pH of from about 6.0 to about 12.0, or from about 7.0 to about 12.0, or at a pH of from about 6 to about 10, or at a pH of from about 6 to about 9. In some embodiments, the polypeptides have nuclease activity at a pH above 6.0, 6.5, 7.0, 7.5, 8.0, 8.5, 9.0, 9.5, 10.0, 10.5, 11.0 or 11.5. In some embodiments, the polypeptides have nuclease activity at a pH below 12.0, 11.5, 11.0, 10.5, 10.0, 9.5, 9.0, 8.5, 8.0, 7.5, 7.0, or 6.5.

In some embodiments, the polypeptides of the present disclosure have nuclease activity at a temperature range from about 10° C. to about 90° C., or from about 20° C. to about 40° C. In some embodiments, the polypeptides of the present disclosure have nuclease activity at a temperature range of from about 20° C. to about 40° C. In some embodiments, the polypeptides have at least 50%, 60%, 70%, 80% or 90% of maximal nuclease activity at a temperature of from about 20° C. to about 40° C. In some embodiments, the polypeptides have activity at a temperature above 50° C., 55° C., 60° C., 65° C., or 70° C. In some embodiments, the polypeptides have activity at a temperature below 90° C., 85° C., 80° C., 75° C., 70° C., 65° C., 60° C., or 55° C.

A nuclease polypeptides of the present disclosure can be subject to various changes, such as one or more amino acid insertions, deletions, and/or substitutions, either conservative or non-conservative, including where such changes do not substantially alter the enzymatic activity of the polypeptide. Similarly, a nucleic acid of the invention can also be subject to various changes, such as one or more substitutions of one or more nucleotides in one or more codons such that a particular codon encodes the same or a different amino acid, resulting in either a silent variation (e.g., when the encoded amino acid is not altered by the nucleotide mutation) or non-silent variation, one or more deletions of one or more nucleic acids (or codons) in the sequence, one or more additions or insertions of one or more nucleic acids (or codons) in the sequence, and/or cleavage of or one or more truncations of one or more nucleic acids (or codons) in the sequence. Many such changes in the nucleic acid sequence may not substantially alter the enzymatic activity of the resulting encoded polypeptide enzyme compared to the polypeptide enzyme encoded by the original nucleic acid sequence. A nucleic acid sequence of the invention can also be modified to include one or more codons that provide for optimum expression in an expression system (e.g., bacterial expression system), while, if desired, said one or more codons still encode the same amino acid(s).

The disclosure provides isolated, non-naturally occurring, or recombinant nucleic acids which may be collectively referred to as “nucleic acids” or “polynucleotides”, which encode polypeptides having an amino acid sequence having at least 50%, 55%, 60%, 65%, 70%, 75%, 80%, 85%, 90%, 91%, 92%, 93%, 94%, 95%, 96%, 97%, 98%, 99%, or 100% sequence identity with the amino acid sequence selected from the group consisting of SEQ ID NOs: 1-13. Nucleic acids of the disclosure, including all described below, are useful in recombinant production (e.g., expression) of polypeptides of the disclosure, typically through expression of a plasmid expression vector comprising a sequence encoding the polypeptide of interest or fragment thereof. As discussed above, polypeptides of the present disclosure include polypeptides having enzymatic activity (e.g., nuclease activity) which are useful in cleaning applications and cleaning compositions for cleaning an item or a surface (e.g., surface of an item) in need of cleaning and/or reducing, removing or preventing biofilms, or for removing microorganisms from an item, a surface, or a solution.

In some embodiments, the polynucleotide of the present disclosure is a polynucleotide having a specified degree of nucleic acid homology to the exemplified polynucleotide. In some embodiments, the polynucleotide has a nucleic acid sequence that encodes a polypeptide or an active fragment thereof having at least 70, 75, 80, 85, 90, 91, 92, 93, 94, 95, 96, 97, 98, 99, or 100% amino acid sequence identity to an amino acid sequence selected from the group consisting of SEQ ID NOs: 1-13. Homology can be determined by amino acid sequence alignment, e.g., using a program such as BLAST, ALIGN, or CLUSTAL, as described herein.

In some embodiments, the disclosure provides an isolated, recombinant, substantially pure, synthetically derived, or non-naturally occurring nucleic acid comprising a nucleotide sequence encoding any polypeptide (including any fusion protein, etc.) having nuclease activity described herein. The disclosure also provides an isolated, recombinant, substantially pure, synthetically derived, or non-naturally-occurring nucleic acid comprising a nucleotide sequence encoding a combination of two or more of any polypeptides provided herein. The present disclosure provides nucleic acids encoding a polypeptide having nuclease activity of the present disclosure, wherein the polypeptide is a mature form having nuclease activity. In some embodiments, the polypeptide is expressed recombinantly with a homologous pro-peptide sequence. In other embodiments, the polypeptide is expressed recombinantly with a heterologous pro-peptide sequence.

The nucleic acids provided herein can be generated by using any suitable synthesis, manipulation, and/or isolation techniques, or combinations thereof. For example, a polynucleotide provided herein may be produced using standard nucleic acid synthesis techniques, such as solid-phase synthesis techniques that are well-known to those skilled in the art. In such techniques, fragments of up to 50 or more nucleotide bases are typically synthesized, then joined (e.g., by enzymatic or chemical ligation methods) to form essentially any desired continuous nucleic acid sequence. The synthesis of the nucleic acids can be also facilitated by any suitable method known in the art, including but not limited to chemical synthesis using the classical phosphoramidite method (See e.g., Beaucage et al. Tetrahedron Letters 22:1859-69 [1981]); or the method described by Matthes et al. (See, Matthes et al., EMBO J. 3:801-805 [1984], as is typically practiced in automated synthetic methods. Nucleic acids of the invention also can be produced by using an automatic DNA synthesizer. Customized nucleic acids can be ordered from a variety of commercial sources (e.g., The Midland Certified Reagent Company, the Great American Gene Company, Operon Technologies Inc., and DNA2.0). Other techniques for synthesizing nucleic acids and related principles are known in the art (See e.g., Itakura et al., Ann. Rev. Biochem. 53:323 [1984]; and Itakura et al., Science 198:1056 [1984]).

The present disclosure also provides recombinant vectors comprising at least one polynucleotide described herein (e.g., a polynucleotide encoding a polypeptide having nuclease activity provided herein), expression vectors or expression cassettes comprising at least one nucleic acid or polynucleotide of the disclosure, isolated, substantially pure, or recombinant DNA constructs comprising at least one nucleic acid or polynucleotide of the disclosure, isolated or recombinant cells comprising at least one polynucleotide of the disclosure, and compositions comprising one or more such vectors, nucleic acids, expression vectors, expression cassettes, DNA constructs, cells, cell cultures, or any combination or mixtures thereof.

In some embodiments, the disclosure provides recombinant cells comprising at least one vector (e.g., expression vector or DNA construct) which comprises at least one nucleic acid or polynucleotide provided herein. Some such recombinant cells are transformed or transfected with such at least one vector, although other methods are available and known in the art. Such cells are typically referred to as host cells. Some such cells comprise bacterial cells, including, but are not limited to Bacillus sp. cells, such as B. subtilis cells. Some such cells comprise fungal cells, including but not limited to Trichoderma cells, such as Trichoderma reesei cells. The disclosure also provides recombinant cells (e.g., recombinant host cells) comprising at least one polypeptide having nuclease activity of the disclosure.

In some embodiments, the disclosure provides a vector comprising a nucleic acid or polynucleotide as described herein. In some embodiments, the vector is an expression vector or expression cassette in which a polynucleotide sequence which encodes a polypeptide having nuclease activity is operably linked to one or additional nucleic acid segments required for efficient gene expression (e.g., a promoter operably linked to the polynucleotide of the invention which encodes a serine protease polypeptide of the invention). A vector may include a transcription terminator and/or a selection gene, such as an antibiotic resistance gene, that enables continuous cultural maintenance of plasmid-infected host cells by growth in antimicrobial-containing media.

An expression vector may be derived from plasmid or viral DNA, or in alternative embodiments, contains elements of both. Exemplary vectors include, but are not limited to pC194, pJH101, pE194, pHP13 (See, Harwood and Cutting [eds.], Chapter 3, Molecular Biological Methods for Bacillus, John Wiley & Sons [1990]; suitable replicating plasmids for B. subtilis include those listed on p. 92) See also, Perego, Integrational Vectors for Genetic Manipulations in Bacillus subtilis, in Sonenshein et al., [eds.] Bacillus subtilis and Other Gram-Positive Bacteria: Biochemistry, Physiology and Molecular Genetics, American Society for Microbiology, Washington, D.C. [1993], pp. 615-624), and p2JM103BBI.

For expression and production of a protein of interest (e.g., a polypeptide having nuclease activity) in a cell, at least one expression vector comprising at least one copy of a polynucleotide encoding the polypeptide having nuclease activity, and in some instances comprising multiple copies, is transformed into the cell under conditions suitable for expression of the polypeptide. In some embodiments, a polynucleotide sequence encoding the polypeptide having nuclease activity (as well as other sequences included in the vector) is integrated into the genome of the host cell, while in other embodiments, a plasmid vector comprising a polynucleotide sequence encoding the polypeptide having nuclease activity remains as autonomous extra-chromosomal clement within the cell. The disclosure provides both extrachromosomal nucleic acid elements as well as incoming nucleotide sequences that are integrated into the host cell genome. The vectors described herein are useful for production of the polypeptides having nuclease activity as provided herein. In some embodiments, a polynucleotide construct encoding the polypeptide is present on an integrating vector that enables the integration and optionally the amplification of the polynucleotide encoding the polypeptide into the host chromosome. Examples of sites for integration are well known to those skilled in the art. In some embodiments, transcription of a polynucleotide encoding a polypeptide of the disclosure is effectuated by a promoter that is the wild-type promoter for the selected precursor nuclease. In some other embodiments, the promoter is heterologous to the precursor nuclease, but is functional in the host cell. Specifically, examples of suitable promoters for use in bacterial host cells include, but are not limited to, for example, the amyE, amyQ, amyL, pstS, sacB, pSPAC, pAprE, pVeg, pHpaII promoters, the promoter of the B. stearothermophilus maltogenic amylase gene, the B. amyloliquefaciens (BAN) amylase gene, the B. subtilis alkaline protease gene, the B. clausii alkaline protease gene the B. pumilis xylosidase gene, the B. thuringiensis cryIIIA, and the B. licheniformis alpha-amylase gene. Additional promoters include, but are not limited to the A4 promoter, as well as phage Lambda PR or PL promoters, and the E. coli lac, trp or tac promoters.

The polypeptides of the present disclosure can be produced in host cells of any suitable microorganism, including bacteria and fungi. In some embodiments, the polypeptides of the present disclosure can be produced in Gram-positive bacteria. In some embodiments, the host cells are Bacillus spp., Streptomyces spp., Escherichia spp., Aspergillus spp., Trichoderma spp., Pseudomonas spp., Corynebacterium spp., Saccharomyces spp., or Pichia spp. In some embodiments, the polypeptides are produced by Bacillus sp. host cells. Examples of Bacillus sp. host cells that find use in the production of the polypeptides of the invention include, but are not limited to B. licheniformis, B. lentus, B. subtilis, B. amyloliquefaciens, B. lentus, B. brevis, B. stearothermophilus, B. alkalophilus, B. coagulans, B. circulans, B. pumilis, B. thuringiensis, B. clausii, and B. megaterium, as well as other organisms within the genus Bacillus. In some embodiments, B. subtilis host cells are used for production of the polypeptides having nuclease activity. U.S. Pat. Nos. 5,264,366 and 4,760,025 (RE 34,606) describe various Bacillus host strains that can be used for producing the polypeptide of the disclosure, although other suitable strains can be used.

Several bacterial strains that can be used to produce polypeptides of the disclosure include non-recombinant (i.e., wild-type) Bacillus sp. strains, as well as variants of naturally-occurring strains and/or recombinant strains. In some embodiments, the host strain is a recombinant strain, wherein a polynucleotide encoding a polypeptide of interest has been introduced into the host. In some embodiments, the host strain is a B. subtilis host strain and particularly a recombinant B. subtilis host strain. Numerous B. subtilis strains are known, including, but not limited to for example, 1A6 (ATCC 39085), 168 (1A01), SB19, W23, Ts85, B637, PB1753 through PB1758, PB3360, JH642, 1A243 (ATCC 39,087), ATCC 21332, ATCC 6051, MI113, DE100 (ATCC 39,094), GX4931, PBT 110, and PEP 211strain (See e.g., Hoch et al., Genetics 73:215-228 [1973]; See also, U.S. Pat. Nos. 4,450,235 and 4,302,544, and EP 0134048, each of which is incorporated by reference in its entirety). The use of B. subtilis as an expression host cell is well known in the art (See e.g., Palva et al., Gene 19:81-87 [1982]; Fahnestock and Fischer, J. Bacteriol., 165:796-804 [1986]; and Wang et al., Gene 69:39-47 [1988]).

In some embodiments, the Bacillus host cell is a Bacillus sp. that includes a mutation or deletion in at least one of the following genes, degU, degS, degR and degQ. In some embodiments, the mutation is in a degU gene, and in some embodiments the mutation is degU(Hy)32 (See e.g., Msadek et al., J. Bacteriol. 172:824-834 [1990]; and Olmos et al., Mol. Gen. Genet. 253:562-567 [1997]). In some embodiments, the Bacillus host comprises a mutation or deletion in scoC4 (See e.g., Caldwell et al., J. Bacteriol. 183:7329-7340 [2001]); spoIIE (See e.g., Arigoni et al., Mol. Microbiol. 31:1407-1415 [1999]); and/or oppA or other genes of the opp operon (See e.g., Perego et al., Mol. Microbiol. 5:173-185 [1991]). Indeed, it is contemplated that any mutation in the opp operon that causes the same phenotype as a mutation in the oppA gene will find use in some embodiments of the altered Bacillus strain. In some embodiments, these mutations occur alone, while in other embodiments, combinations of mutations are present. In some embodiments, an altered Bacillus host cell strain that can be used to produce a nuclease polypeptide of the invention is a Bacillus host strain that already includes a mutation in one or more of the above-mentioned genes. In addition, Bacillus sp. host cells that comprise mutation(s) and/or deletions of endogenous protease genes find use. In some embodiments, the Bacillus host cell comprises a deletion of the aprE and the nprE genes. In other embodiments, the Bacillus sp. host cell comprises a deletion of 5 protease genes, while in other embodiments, the Bacillus sp. host cell comprises a deletion of 9 protease genes (See e.g., US 2005/0202535, incorporated herein by reference).

Host cells are transformed with at least one nucleic acid encoding at least one nuclease polypeptide of the invention using any suitable method known in the art. Methods for introducing a nucleic acid (e.g., DNA) into Bacillus cells or E. coli cells utilizing plasmid DNA constructs or vectors and transforming such plasmid DNA constructs or vectors into such cells are well known. In some embodiments, the plasmids are subsequently isolated from E. coli cells and transformed into Bacillus cells. However, it is not essential to use intervening microorganisms such as E. coli, and in some embodiments, a DNA construct or vector is directly introduced into a Bacillus host.

Suitable methods for introducing nucleic acid sequences of the invention into Bacillus cells include those described, for example, in Ferrari et al., “Genetics,” in Harwood et al. [eds.], Bacillus, Plenum Publishing Corp. [1989], pp. 57-72; Saunders et al., J. Bacteriol. 157:718-726 [1984]; Hoch et al., J. Bacteriol. 93:1925-1937 [1967]; Mann et al., Current Microbiol. 13:131-135 [1986]; Holubova, Folia Microbiol. 30:97 [1985]; Chang et al., Mol. Gen. Genet. 168:11-115 [1979]; Vorobjeva et al., FEMS Microbiol. Lett. 7:261-263 [1980]; Smith et al., Appl. Env. Microbiol. 51:634 [1986]; Fisher et al., Arch. Microbiol. 139:213-217 [1981]; and McDonald, J. Gen. Microbiol. 130:203 [1984]). Indeed, such methods as transformation, including protoplast transformation and transfection, transduction, and protoplast fusion are well known and suited for use in the present invention. Methods known in the art to transform Bacillus cells include such methods as plasmid marker rescue transformation, which involves the uptake of a donor plasmid by competent cells carrying a partially homologous resident plasmid (See, Contente et al., Plasmid 2:555-571 [1979]; Haima et al., Mol. Gen. Genet. 223:185-191 [1990]; Weinrauch et al., J. Bacteriol. 154:1077-1087 [1983]; and Weinrauch et al., J. Bacteriol. 169:1205-1211 [1987]). In this method, the incoming donor plasmid recombines with the homologous region of the resident “helper” plasmid in a process that mimics chromosomal transformation.

In addition to commonly used methods, in some embodiments, host cells are directly transformed with a DNA construct or vector comprising a nucleic acid encoding a nuclease polypeptide of the invention (i.e., an intermediate cell is not used to amplify, or otherwise process, the DNA construct or vector prior to introduction into the host cell). Introduction of the DNA construct or vector of the invention into the host cell includes those physical and chemical methods known in the art to introduce a nucleic acid sequence (e.g., DNA sequence) into a host cell without insertion into the host genome. Such methods include, but are not limited to calcium chloride precipitation, electroporation, naked DNA, liposomes and the like. In additional embodiments, DNA constructs or vector are co-transformed with a plasmid, without being inserted into the plasmid. In further embodiments, a selective marker is deleted from the altered Bacillus strain by methods known in the art (See, Stahl et al., J. Bacteriol. 158:411-418 [1984]; and Palmeros et al., Gene 247:255-264 [2000]).

In some embodiments, the transformed cells of the present invention are cultured in conventional nutrient media. The suitable specific culture conditions, such as temperature, pH and the like are known to those skilled in the art and are well described in the scientific literature. In some embodiments, the invention provides a culture (e.g., cell culture) comprising at least one nuclease polypeptide or at least one nucleic acid of the disclosure.

In some embodiments, host cells transformed with at least one polynucleotide sequence encoding at least one nuclease polypeptide of the disclosure are cultured in a suitable nutrient medium under conditions permitting the expression of the present nuclease, after which the resulting nuclease is recovered from the culture. In some embodiments, the nuclease produced by the cells is recovered from the culture medium by conventional procedures, including, but not limited to for example, separating the host cells from the medium by centrifugation or filtration, precipitating the proteinaceous components of the supernatant or filtrate by means of a salt (e.g., ammonium sulfate), chromatographic purification (e.g., ion exchange, gel filtration, affinity, etc.).

In some embodiments, a nuclease polypeptide produced by a recombinant host cell is secreted into the culture medium. A nucleic acid sequence that encodes a purification facilitating domain may be used to facilitate purification of proteins. A vector or DNA construct comprising a polynucleotide sequence encoding a nuclease polypeptide may further comprise a nucleic acid sequence encoding a purification facilitating domain to facilitate purification of the nuclease polypeptide (See e.g., Kroll et al., DNA Cell Biol. 12:441-53 [1993]). Such purification facilitating domains include, but are not limited to, for example, metal chelating peptides such as histidine-tryptophan modules that allow purification on immobilized metals (See, Porath, Protein Expr. Purif. 3:263-281 [1992]), protein A domains that allow purification on immobilized immunoglobulin, and the domain utilized in the FLAGS extension/affinity purification system. The inclusion of a cleavable linker sequence such as Factor XA or enterokinase (e.g., sequences available from Invitrogen, San Diego, CA) between the purification domain and the heterologous protein also find use to facilitate purification.

Assays for detecting and measuring the enzymatic activity of an enzyme, such as a nuclease polypeptide of the invention, are well known. Various assays for detecting and measuring activity of nucleases, are also known to those of ordinary skill in the art. In particular, assays are available for measuring nuclease activity such as those described in the examples, or such as by monitoring the hydrolysis of DNA fragments of known size with gel electrophoresis and visualization with ethidium bromide or other stain, or such as by the use of a commercial kit (e.g. abcam DNAse I Assay Kit), or such as by the use of a published nuclease activity determination method (e.g., Sinicropi, D., Baker, D. L., Prince, W. S., Shiffer, K., Shak, S. (1994) Colorimetric determination of DNase I activity with a DNA-methyl green substrate. Analytica Biochemistry 222: 351-358; Nass, K, Frenkel, G. D. (1978) Adenovirus-induced inhibition of cellular DNAse. Journal of Virology 26: 540-543.; Shak, S., Capon, D. J., Hellmiss, R., Marsters, S. A., Baker, C. L. (1990) Recombinant human DNAse I reduces the viscosity of cystic fibrosis sputum, Proceedings of the National Academy of Sciences USA 87: 9188-9192; Nadano, D., Yasuda, T., Kishi, K. (1991) Purification and characterization of genetically polymorphic deoxyribonuclease I from human kidney. Journal of Biochemistry 110: 321-323.)

A variety of methods can be used to determine the level of production of a mature nuclease in a host cell. Such methods include, but are not limited to, for example, methods that utilize either polyclonal or monoclonal antibodies specific for the nuclease. Exemplary methods include but are not limited to enzyme-linked immunosorbent assays (ELISA), radioimmunoassays (RIA), fluorescent immunoassays (FIA), and fluorescent activated cell sorting (FACS). These and other assays are well known in the art (See e.g., Maddox et al., J. Exp. Med. 158:1211 [1983]).

In some other embodiments, the invention provides methods for making or producing a mature nuclease polypeptide of the disclosure. A mature nuclease polypeptide does not include a signal peptide or a propeptide sequence. Some methods comprise making or producing a nuclease polypeptide of the disclosure in a recombinant bacterial host cell, such as for example, a Bacillus sp. cell (e.g., a B. subtilis cell). In some embodiments, the disclosure provides a method of producing a nuclease polypeptide of the invention, the method comprising cultivating a recombinant host cell comprising a recombinant expression vector comprising a nucleic acid encoding a nuclease polypeptide of the disclosure (e.g. a polypeptide having at least 70%, 75%, 80%, 85%, 90%, 95%, 96%, 97%, 98%, 99%, or 100% to an amino acid sequence of SEQ ID NOs: 1-13) under conditions conducive to the production of the nuclease polypeptide. Some such methods further comprise recovering the nuclease polypeptide from the culture.

In some embodiments the disclosure provides methods of producing a nuclease polypeptide of the invention, the methods comprising: (a) introducing a recombinant expression vector comprising a nucleic acid encoding a nuclease polypeptide of the disclosure (e.g. a polypeptide having at least 50%, 55%, 60%, 65%, 70%, 75%, 80%, 85%, 90%, 95%, 96%, 97%, 98%, 99%, or 100% to an amino acid sequence of SEQ ID NOs: 1-13) into a population of cells (e.g., bacterial cells, such as B. subtilis cells); and (b) culturing the cells in a culture medium under conditions conducive to produce the nuclease polypeptide encoded by the expression vector. Some such methods further comprise: (c) isolating the nuclease polypeptide from the cells or from the culture medium.

### Methods

In one embodiment, the invention provides a method for preventing, reducing or removing a biofilm comprising contacting the biofilm with a cleaning composition comprising a polypeptide having nuclease activity, wherein the polypeptide having the nuclease activity is a polypeptide having at least 80% sequence identity with an enzyme selected from the group consisting of SEQ ID NO: 1 to 13. The described biofilm is on a textile or hard surface. The hard surface is selected from the group consisting of a laundry machine surface, a dish surface, or a dishwasher surface. The cleaning composition comprises a polypeptide having nuclease activity in an amount selected from 0.001 to 10,000 mg/L, or 0.001 to 2000 mg/L, or 0.01 to 5000 mg/L, or 0.01 to 2000 mg/L, or 0.01 to 1300 mg/L, or 0.1 to 5000 mg/L, or 0.1 to 2000 mg/L, or 0.1 to 1300 mg/L, or 1 to 5000 mg/L, or J to 1300 mg/L, or 1 to 500 mg/L, or 10 to 5000 mg/L, or 10 to 1300 mg/L, or 10 to 500 mg/L. The cleaning composition is a laundry composition.

In one embodiment, the invention is also a method for preventing, reducing or removing a biofilm from a textile or hard surface comprising: (i) contacting a textile or surface with a polypeptide having nuclease activity or a composition comprising a polypeptide having nuclease activity; and (ii) optionally, rinsing the textile or surface, wherein the polypeptide having the nuclease activity is a polypeptide having at least 80% sequence identity selected from the group consisting of SEQ ID NO: 1 to 13. The textile comprises a biofilm on a surface of the textile. The biofilm is reduced or removed from the textile. The biofilm is reduced or removed from the article in an amount selected from the groups consisting of at least 5%, 10%, 15%, 20%, 25%, 30%, 35%, 40%, 45%, 50%, 55%, 60%, 65%, 70%, 75%, 80%, 85%, 90%, 95% or greater compared to the amount of the biofilm present on the textile prior to contacting the textile with the polypeptide having nuclease activity or a composition comprising a polypeptide having nuclease activity. The biofilm is measured by staining with crystal violet, using the method of Example 2. Further, the contacting step comprises the use a polypeptide having nuclease activity in an amount selected from the group consisting of 0.002 to 10,000 mg of protein, 0.005 to 5000 mg of protein. 0.01 to 5000 mg of protein, 0.05 to 5000 mg of protein, 0.05 to 1300 mg of protein, 0.1 to 1300 mg of protein, 0.1 to 500 mg of protein, 0.1 to 100 mg of protein, per liter of wash liquor, or in the amount of at least 0.002 ppm active nuclease. This cleaning composition has a pH of from 7.4 to pH 11.5, or pH 7.4 to pH 11.0, or pH 7.5 to pH 11.5. The contacting step occurs in a wash liquor, and the contacting step takes place for an amount of time selected from the group consisting of about 5 minutes to about 10 days, about 5 minutes to about 400 minutes, between about 5 minutes to about 300 minutes, between about 5 minutes to about 250 minutes, between about 5 minutes to about 200 minutes, between about 5 minutes to about 150 minutes, between about 5 minutes to about 100 minutes, between about 5 minutes to about 50 minutes, between about 5 minutes to about 30 minutes. The contacting step takes place at a temperature selected from the group consisting of about 10° to 60° C., between 15° to about 55° C., between 20° to about 50° C. and between 20° to about 45° C.

In one embodiment, the invention is a method for preventing, reducing or removing a biofilm comprising contacting the biofilm with a cleaning composition comprising a polypeptide having nuclease activity and at least one additional enzyme selected from the group consisting of DNase, acyl transferases, alpha-amylases, beta-amylases, alpha-galactosidases, arabinosidases, aryl esterases, beta-galactosidases, carrageenases, catalases, cellobiohydrolases, cellulases, chondroitinases, cutinases, endo-beta-1,4-glucanases, endo-beta-mannanases, esterases, exo-mannanases, galactanases, glucoamylases, hemicellulases, hyaluronidases, keratinases, laccases, lactases, ligninases, lipases, lipoxygenases, lysozymes, mannanases, metalloproteases, nucleases, oxidases, oxidoreductases, pectate lyases, pectin acetyl esterases, pectinases, pentosanases, peroxidases, phenoloxidases, phosphatases, phospholipases, phytases, polygalacturonases, polyesterases, proteases, pullulanases, reductases, rhamnogalacturonases, beta-glucanases, tannases, transglutaminases, xylan acetyl-esterases, xylanases, xyloglucanases, xylosidases, and any combination or mixture thereof.

The invention is a method for preventing, reducing or removing a biofilm comprising contacting the biofilm with a cleaning composition comprising a polypeptide having nuclease activity and a lysozyme. The invention is also a detergent composition comprising: (i) a polypeptide having nuclease activity and (ii) a lysozyme.

### Compositions

The composition comprising a polypeptide having nuclease activity further comprises a surfactant. The surfactant is selected from the group consisting of a non-ionic, ampholytic, semi-polar, anionic, cationic, zwitterionic, and combinations and mixtures thereof. The composition is a detergent composition. The composition may comprise one or more additional enzymes selected from the group consisting of acyl transferases, alpha-amylases, beta-amylases, alpha-galactosidases, arabinosidases, aryl esterases, beta-galactosidases, carrageenases, catalases, cellobiohydrolases, cellulases, chondroitinases, cutinases, endo-beta-1,4-glucanases, endo-beta-mannanases, esterases, exo-mannanases, galactanases, glucoamylases, hemicellulases, hyaluronidases, keratinases, laccases, lactases, ligninases, lipases, lipoxygenases, lysozymes, mannabases, metalloproteases, oxidases, oxidoreductases, pectate lyases, pectin acetyl esterases, pectinases, pentosanases, peroxidases, phenoloxidases, phosphatases, phospholipases, phytases, polygalacturonases, polyesterases, proteases, pullulanases, reductases, rhamnogalacturonases, beta-glucanases, tannases, transglutaminases, xylan acetyl-esterases, xylanases, xyloglucanases, xylosidases, and any combination or mixture thereof. Further, the contacting step takes place in a washing machine or a dishwasher.

In one embodiment, the invention is a detergent composition comprising: (i) a polypeptide having nuclease activity; (ii) a polypeptide having protease activity; (iii) at least one additional polypeptide, wherein the at least one additional polypeptide is an enzyme selected from: DNaseacyl transferases, alpha-amylases, beta-amylases, alpha-galactosidases, arabinosidases, aryl esterases, beta-galactosidases, carrageenases, catalases, cellobiohydrolases, cellulases, chondroitinases, cutinases, endo-beta-1,4-glucanases, endo-beta-mannanases, esterases, exo-mannanases, galactanases, glucoamylases, hemicellulases, hyaluronidases, keratinases, laccases, lactases, ligninases, lipases, lipoxygenases, lysozymes, mannanases, metalloproteases, nucleases, oxidases, oxidoreductases, pectate lyases, pectin acetyl esterases, pectinases, pentosanases, peroxidases, phenoloxidases, phosphatases, phospholipases, phytases, polygalacturonases, polyesterases, proteases, pullulanases, reductases, rhamnogalacturonases, beta-glucanases, tannases, transglutaminases, xylan acetyl-esterases, xylanases, xyloglucanases, xylosidases, and any combination or mixture thereof; and (iv) a surfactant, wherein the polypeptide having the nuclease activity is a polypeptide having at least 80% sequence identity selected from the group consisting of SEQ ID NO: 1 to 13. The surfactant is selected from the group consisting of a non-ionic, ampholytic, semi-polar, anionic, cationic, zwitterionic, and combinations and mixtures thereof. The composition further comprises between about 0.1% to about 60%, about 1% to about 50%, or about 5% to about 40% surfactant by weight of the composition. The composition further comprises one or more adjunct materials selected from the group consisting of builders, bleaches, bleach activators, bleach catalysts, other enzymes, enzyme stabilizing systems, chelants, optical brighteners, soil release polymers, dye transfer agents, dispersants, suds suppressors, dyes, perfumes, colorants, filler salts, hydrotropes, photoactivators, fluorescers, fabric conditioners, hydrolyzable surfactants, preservatives, anti-oxidants, anti-shrinkage agents, anti-wrinkle agents, germicides, fungicides, color speckles, silvercare, anti-tarnish and/or anti-corrosion agents, alkalinity sources, solubilizing agents, carriers, processing aids, pigments, and pH control agents. The nuclease described is a DNase.

### Malodor Reduction

In one embodiment, the invention is a method for reducing malodor associated with a textile or hard surface comprising: (i) contacting a textile or hard surface with a polypeptide having nuclease activity or a composition comprising a polypeptide having nuclease activity; and (ii) optionally, rinsing the textile or surface, wherein the polypeptide having the nuclease activity is a polypeptide having at least 80% sequence identity selected from a group consisting of SEQ ID NO: 1 to 13. The textile comprises a biofilm on a surface of the textile or hard surface. Further, the biofilm is reduced or removed from the textile. Further, the malodor is reduced at least 5%, 10%, 15%, 20%, 25%, 30%, 35%, 40%, 45%, 50%, 55%, 60%, 65%, 70%, 75%, 80%, 85%, 90%, 95% or greater compared to the amount of the malodor present prior to contacting the textile or hard surface with the polypeptide having nuclease activity or a composition comprising a polypeptide having nuclease activity. The contacting step comprises the use a polypeptide having nuclease activity in an amount selected from the group consisting of 0.002 to 10,000 mg of protein, 0.005 to 5000 mg of protein, 0.01 to 5000 mg of protein. 0.05 to 5000 mg of protein. 0.05 to 1300 mg of protein, 0.1 to 1300 mg of protein, 0.1 to 500 mg of protein, 0.1 to 100 mg of protein, per liter of wash liquor, or in the amount of at least 0.002 ppm active nuclease. The polypeptide having nuclease activity is a polypeptide having at least 85%, 90%, 95%, 96%, 97%, 98%, 99%, or 100% sequence identity selected from a group consisting of SEQ ID NO: 1 to 13. The contacting step occurs in a wash liquor. Further, the contacting step takes place at a temperature selected from the group consisting of about 10° to 60° C., between 15° to about 55° C., between 20° to about 50° C. and between 20° to about 45° C. Further, the composition comprising a polypeptide having nuclease activity further comprises a surfactant. The surfactant is selected from the group consisting of a non-ionic, ampholytic, semi-polar, anionic, cationic, zwitterionic, and combinations and mixtures thereof. The composition described is a detergent composition. The contacting step further includes contacting the textile with one or more additional enzymes selected from the group consisting of acyl transferases, alpha-amylases, beta-amylases, alpha-galactosidases, arabinosidases, aryl esterases, beta-galactosidases, carrageenases, catalases, cellobiohydrolases, cellulases, chondroitinases, cutinases, endo-beta-1,4-glucanases, endo-beta-mannanases, esterases, exo-mannanases, galactanases, glucoamylases, hemicellulases, hyaluronidases, keratinases, laccases, lactases, ligninases, lipases, lipoxygenases, lysozymes, mannanases, metalloproteases, oxidases, oxidoreductases, pectate lyases, pectin acetyl esterases, pectinases, pentosanases, peroxidases, phenoloxidases, phosphatases, phospholipases, phytases, polygalacturonases, polyesterases, proteases, pullulanases, reductases, rhamnogalacturonases, beta-glucanases, tannases, transglutaminases, xylan acetyl-esterases, xylanases, xyloglucanases, xylosidases, and any combination or mixture thereof. Further, the contacting step takes place in a washing machine or a dishwasher.

In one embodiment, the invention is an isolated polypeptide or active fragment thereof having nuclease activity, wherein the polypeptide comprises an amino acid sequence having at least 75%. 80%, 85%, 90%, 91%, 92%, 93%, 94%, 95%, 96%, 97%, 98%, 99%, or 100% sequence identity with an amino acid sequence selected from the group consisting of SEQ ID NO: 1 to 13.

### Cleaning Methods

The polypeptides, compositions, and methods provided herein have utility in a wide array of applications in which preventing, reducing, or removing biofilms is desired, such as household cleaning, including in washing machines, dishwashers, and on household surfaces. The polypeptides, compositions, and methods also have applications in treating medical and dental biofilms, including but not limited to plaque on teeth, lung infections, on catheters and implanted medical devices, on contact lenses, in medical instrument cleaning, and in wound healing. The polypeptides, compositions, and methods provided herein can also be used to treat biofouling in various industrial settings, including but not limited to in oil and gas recovery, water treatment facilities, in marine equipment, in animal care settings, and in food preservation.

An embodiment is directed to a method of laundering a textile, where the method comprises contacting a textile with a polypeptide having nuclease activity, or a composition comprising a polypeptide having nuclease activity for an amount of time sufficient to prevent, reduce or remove a biofilm from the textile and optionally rinsing the textile.

An embodiment is directed to a method for cleaning an article, where the method comprises contacting the article with a polypeptide having nuclease activity or a composition having a polypeptide having nuclease activity under conditions sufficient reduce or remove a biofilm from the article, and optionally rinsing the article.

In another embodiment, the prevention or reduction of a biofilm includes the reduction in the formation, growth, or proliferation of biofilm on a textile or hard surface. In one embodiment, the reduction in the formation, growth, or proliferation of biofilm on a textile or hard surface may be measured by following the change in the amount of the biofilm over a suitable time period with the method provided in Examples, 3, 2 4 and 5 below, or another suitable method in the art. For example, biofilm formation or growth may be inhibited in an amount ranging from 1% to about 99% relative to that of an untreated hard surface or textile. Biofilm formation may be inhibited by at least about 10%, at least about 20%, at least about 30%, at least about 40%, at least about 50%, at least about 60%, at least about 70%, at least about 80%, at least about 90%, or at least about 95% relative to biofilm formation on an untreated hard surface or textile. In another embodiment, the formation of biofilm on a surface may be delayed over a number of laundry cycles (e.g. 1, 2, 3, 4, 5, or more cycles), compared to that of an untreated surface.

The textile or surface can be contacted with the polypeptide or a composition comprising the polypeptide having nuclease activity in a washing machine or in a manual wash tub (e.g. for handwashing). In one embodiment, the textile or surface is contacted with the polypeptide having nuclease activity or the composition comprising a peptide having nuclease activity in a wash liquor. In another embodiment, a solution containing the polypeptide having nuclease activity is incubated with or flowed over the hard surface, such as by pumping the solution through tubing or pipes or by filling a reservoir with the solution.

The nuclease polypeptide for use in the methods and compositions herein includes any nuclease polypeptide. As used herein, “homologous genes” refers to a pair of genes from different, but usually related species, which correspond to each other and which are identical or very similar to each other. The term encompasses genes that are separated by speciation (i.e., the development of new species) (e.g., orthologous genes), as well as genes that have been separated by genetic duplication (e.g., paralogous genes). As used herein, the term “variant polypeptide” refers to a polypeptide comprising an amino acid sequence that differs in at least one amino acid residue from the amino acid sequence of a parent or reference polypeptide (including but not limited to wild-type polypeptides).

As used herein, “the genus Bacillus” includes all species within the genus “Bacillus,” as known to those of skill in the art, including but not limited to B. subtilis, B. licheniformis, B. lentus, B. brevis, B. stearothermophilus, B. alkalophilus, B. amyloliquefaciens, B. clausii, B. halodurans, B. megaterium, B. coagulans, B. circulans, B. lautus, and B. thuringiensis. It is recognized that the genus Bacillus continues to undergo taxonomical reorganization. Thus, it is intended that the genus include species that have been reclassified, including but not limited to such organisms as B. stearothermophilus, which is now named “Geobacillus stearothermophilus.” The production of resistant endospores in the presence of oxygen is considered the defining feature of the genus Bacillus, although this characteristic also applies to the recently named Alicyclobacillus, Amphibacillus, Aneurinibacillus, Anoxybacillus, Brevibacillus, Filobacillus, Gracilibacillus, Halobacillus, Paenibacillus, Salibacillus, Thermobacillus, Ureibacillus, and Virgibacillus.

As used herein, “nuclease activity” refers to a protein or polypeptide exhibiting the ability to hydrolyze the cleavage of phosphodiester linkages in a DNA backbone. Methods for measuring nuclease activity are known, and include those provided herein.

As used herein, “% identity or percent identity” refers to sequence similarity. Percent identity may be determined using standard techniques known in the art (See e.g., Smith and Waterman, Adv. Appl. Math. 2:482 [1981]; Needleman and Wunsch, J. Mol. Biol. 48:443 [1970]; Pearson and Lipman, Proc. Natl. Acad. Sci. USA 85:2444 [1988]; software programs such as GAP, BESTFIT, FASTA, and TFASTA in the Wisconsin Genetics Software Package (Genetics Computer Group, Madison, WI); and Devereux et al., Nucl. Acid Res. 12:387-395 [1984]). One example of a useful algorithm is PILEUP. PILEUP creates a multiple sequence alignment from a group of related sequences using progressive, pair-wise alignments. It can also plot a tree showing the clustering relationships used to create the alignment. PILEUP uses a simplification of the progressive alignment method of Feng and Doolittle (See, Feng and Doolittle, J. Mol. Evol. 35:351-360 [1987]). The method is similar to that described by Higgins and Sharp (See, Higgins and Sharp, CABIOS 5:151-153 [1989]). Useful PILEUP parameters include a default gap weight of 3.00, a default gap length weight of 0.10, and weighted end gaps. Other useful algorithm is the BLAST algorithms described by Altschul et al., (See, Altschul et al., J. Mol. Biol. 215:403-410 [1990]; and Karlin and Altschul, Proc. Natl. Acad. Sci. USA 90:5873-5787 [1993]). The BLAST program uses several search parameters, most of which are set to the default values.

As used herein, “homologous proteins” or “homologous proteases” refers to proteins that have distinct similarity in primary, secondary, and/or tertiary structure. Protein homology can refer to the similarity in linear amino acid sequence when proteins are aligned. Homology can be determined by amino acid sequence alignment, e.g., using a program such as BLAST, MUSCLE, or CLUSTAL. Homologous search of protein sequences can be done using BLASTP and PSI-BLAST from NCBI BLAST with threshold (E-value cut-off) at 0.001. (Altschul et al., “Gapped BLAST and PSI BLAST a new generation of protein database search programs”, Nucleic Acids Res, Set 1;25(17):3389-402(1997)). The BLAST program uses several search parameters, most of which are set to the default values. The NCBI BLAST algorithm finds the most relevant sequences in terms of biological similarity but is not recommended for query sequences of less than 20 residues (Altschul et al., Nucleic Acids Res, 25:3389-3402, 1997 and Schaffer et al., Nucleic Acids Res, 29:2994-3005, 2001). Exemplary default BLAST parameters for a nucleic acid sequence searches include: Neighboring words threshold=11; E-value cutoff=10; Scoring Matrix=NUC.3.1 (match=1, mismatch=−3); Gap Opening=5; and Gap Extension=2. Exemplary default BLAST parameters for amino acid sequence searches include: Word size=3; E-value cutoff=10; Scoring Matrix=BLOSUM62; Gap Opening=11; and Gap extension=1. Using this information, protein sequences can be grouped and/or a phylogenetic tree built therefrom. Amino acid sequences can be entered in a program such as the Vector NTI Advance suite and a Guide Tree can be created using the Neighbor Joining (NJ) method (Saitou and Nei, Mol Biol Evol, 4:406-425, 1987). The tree construction can be calculated using Kimura's correction for sequence distance and ignoring positions with gaps. A program such as AlignX can display the calculated distance values in parentheses following the molecule name displayed on the phylogenetic tree.

A percent (%) amino acid sequence identity value is determined by the number of matching identical residues divided by the total number of residues of the “reference” sequence including any gaps created by the program for optimal/maximum alignment. If a sequence is 90% identical to SEQ ID NO: A, SEQ ID NO: A is the “reference” sequence. BLAST algorithms refer the “reference” sequence as “query” sequence.

The CLUSTAL W algorithm is another example of a sequence alignment algorithm (See, Thompson et al., Nucleic Acids Res, 22:4673-4680, 1994). Default parameters for the CLUSTAL W algorithm include: Gap opening penalty=10.0; Gap extension penalty=0.05; Protein weight matrix=BLOSUM series; DNA weight matrix=IUB; Delay divergent sequences %=40; Gap separation distance=8; DNA transitions weight=0.50; List hydrophilic residues=GPSNDQEKR; Use negative matrix=OFF; Toggle Residue specific penalties=ON; Toggle hydrophilic penalties=ON; and Toggle end gap separation penalty=OFF. In CLUSTAL algorithms, deletions occurring at either terminus are included. For example, a variant with a five amino acid deletion at either terminus (or within the polypeptide) of a polypeptide of 500 amino acids would have a percent sequence identity of 99% (495/500 identical residues×100) relative to the “reference” polypeptide. Such a variant would be encompassed by a variant having “at least 99% sequence identity” to the polypeptide.

Also provided are detergent compositions for use in the methods provided herein. As used herein, the term “detergent composition” or “detergent formulation” is used in reference to a composition intended for use in a wash medium (e.g. a wash liquor) for the cleaning of soiled or dirty objects, including particular textile or non-textile objects or items. Such compositions of the present invention are not limited to any particular detergent composition or formulation. Indeed, in some embodiments, the detergents of the invention comprise at least one nuclease polypeptide and, in addition, one or more surfactants, transferase(s), hydrolytic enzymes, oxido reductases, builders (e.g., a builder salt), bleaching agents, bleach activators, bluing agents, fluorescent dyes, caking inhibitors, masking agents, enzyme activators, antioxidants, and/or solubilizers. In some instances, a builder salt is a mixture of a silicate salt and a phosphate salt, preferably with more silicate (e.g., sodium metasilicate) than phosphate (e.g., sodium tripolyphosphate). Some compositions of the invention, such as, but not limited to, cleaning compositions or detergent compositions, do not contain any phosphate (e.g., phosphate salt or phosphate builder).

The detergent or cleaning compositions of the present invention are advantageously employed for example, in laundry applications, hard surface cleaning, dishwashing applications, as well as cosmetic applications such as dentures, teeth, hair and skin. In addition, due to the unique advantages of increased effectiveness in lower temperature solutions, the enzymes of the present invention are ideally suited for laundry applications. Furthermore, the enzymes of the present invention find use in granular and liquid compositions.

Enzyme component weights are based on total active protein. All percentages and ratios are calculated by weight unless otherwise indicated. All percentages and ratios are calculated based on the total composition unless otherwise indicated. In laundry detergent compositions, the enzyme levels are expressed in ppm, which equals mg active protein/kg detergent composition.

Exemplary surfactants include, but are not limited to sodium dodecylbenzene sulfonate, C12-14 pareth-7, C12-15 pareth-7, sodium C12-15 pareth sulfate, C14-15 pareth-4, sodium laureth sulfate (e.g., Steol CS-370), sodium hydrogenated cocoate, C12 ethoxylates (Alfonic 1012-6, Hetoxol LA7, Hetoxol LA4), sodium alkyl benzene sulfonates (e.g., Nacconol 90G), and combinations and mixtures thereof. Anionic surfactants include but are not limited to linear alkylbenzenesulfonate (LAS), alpha-olefinsulfonate (AOS), alkyl sulfate (fatty alcohol sulfate) (AS), alcohol ethoxysulfate (AEOS or AES), secondary alkanesulfonates (SAS), alpha-sulfo fatty acid methyl esters, alkyl- or alkenylsuccinic acid, or soap. Nonionic surfactants include but are not limited to alcohol ethoxylate (ABO or AE), carboxylated alcohol ethoxylates, nonylphenol ethoxylate, alkylpolyglycoside, alkyldimethylamine oxide, ethoxylated fatty acid monoethanolamide, fatty acid monoethanolamide, polyhydroxy alkyl fatty acid amide (e.g., as described in WO92/06154), polyoxyethylene esters of fatty acids, polyoxyethylene sorbitan esters (e.g., TWEENs), polyoxyethylene alcohols, polyoxyethylene isoalcohols, polyoxyethylene ethers (e.g., TRITONs and BRIJ), polyoxyethylene esters, polyoxyethylene-p-tert-octylphenols or octylphenyl-ethylene oxide condensates (e.g., NONIDET P40), ethylene oxide condensates with fatty alcohols (e.g., LUBROL). polyoxyethylene nonylphenols, polyalkylene glycols (SYNPERONIC F108), sugar-based surfactants (e.g., glycopyranosides, thioglycopyranosides), and combinations and mixtures thereof.

In some embodiments, the compositions provided herein comprise a polypeptide having nuclease activity in combination with a protease. The protease for use in combination with the nuclease in the compositions of the instant disclosure include any polypeptide having protease activity. In one embodiment, the additional protease is a serine protease. In another embodiment, the additional protease is an additional metalloprotease, a fungal subtilisin, or an alkaline microbial protease or a trypsin-like protease. Suitable additional proteases include those of animal, vegetable or microbial origin. In some embodiments, the protease is a microbial protease. In other embodiments, the protease is a chemically or genetically modified mutant. In another embodiment, the protease is subtilisin like protease or a trypsin-like protease. In other embodiments, the additional protease does not contain cross-reactive epitopes with the variant as measured by antibody binding or other assays available in the art. Exemplary subtilisin proteases include those derived from for example, Bacillus (e.g., e.g., BPN′, Carlsberg, subtilisin 309, subtilisin 147, and subtilisin 168), or fungal origin, such as, for example, those described in U.S. Pat. No. 8,362,222. Exemplary additional proteases include but are not limited to those described in WO92/21760, WO95/23221, WO2008/010925, WO09/149200, WO09/149144, WO09/149145, WO 10/056640, WO10/056653, WO2010/0566356, WO11/072099, WO2011/13022, WO11/140364, WO 12/151534, WO2015/038792, WO2015/089447, WO2015/089441, WO 2017/215925, US Publ. No. 2008/0090747, U.S. Pat. Nos. 5,801,039, 5,340,735, 5,500,364, 5,855,625, RE 34,606, 5,955,340, 5,700,676 6,312,936, 6,482,628, 8,530,219, U.S. Provisional Appl Nos. 62/180,673 and 62/1610,77, and PCT Appl Nos. PCT/US2015/021813, PCT/US2015/055900, PCT/US2015/057497, PCT/US2015/057492, PCT/US2015/057512, PCT/US2015/057526, PCT/US2015/057520, PCT/US2015/057502, PCT/US2016/022282, and PCT/US16/32514, International publications WO2016001449, WO2016087617, WO2016096714, WO2016203064, WO2017089093, and WO2019180111, as well as metalloproteases described in WO1999014341, WO1999033960, WO1999014342, WO1999034003, WO2007044993, WO2009058303, WO 2009058661, WO2014071410, WO2014194032, WO2014194034, WO 2014194054, and WO 2014/194117. Exemplary additional proteases include, but are not limited to trypsin (e.g., of porcine or bovine origin) and the Fusarium protease described in WO89/06270. Exemplary commercial proteases include, but are not limited to MAXATASE®, MAXACAL™, MAXAPEM™, OPTICLEAN®, OPTIMASE®, PROPERASE®, PURAFECT®, PURAFECT® OXP, PURAMAX™, EXCELLASE™, PREFERENZ™ proteases (e.g. P100, P110, P280), EFFECTENZ™ proteases (e.g. P1000, P1050, P2000), EXCELLENZ™ proteases (e.g. P1000), ULTIMASE®, and PURAFAST™ (DuPont); ALCALASE®, BLAZE®, BLAZE® variants, BLAZE® EVITY®, BLAZE® EVITY® 16L, CORONASE®, SAVINASE®, SAVINASE® ULTRA, SAVINASE® EVITY®, SAVINASE® EVERIS®, PRIMASE®, DURAZYM™, POLARZYME®, OVOZYME®, KANNASE®, LIQUANASE®, LIQUANASE EVERIS®, NEUTRASE®, PROGRESS UNO®, RELASE®, and ESPERASE® (Novozymes); BLAP™ and BLAP™ variants (Henkel); LAVERGY™ PRO 104 L (BASF), KAP (B. alkalophilus subtilisin (Kao)) and BIOTOUCH® (AB Enzymes).

An exemplary amylase can be a chemically or genetically modified mutant. Exemplary amylases include, but are not limited to those of bacterial or fungal origin, such as, for example, amylases described in GB 1,296,839, WO9100353, WO9402597, WO94183314, WO9510603, WO9526397, WO9535382, WO9605295, WO9623873, WO9623874, WO 9630481, WO9710342, WO9741213, WO9743424, WO9813481, WO 9826078, WO9902702, WO 9909183, WO9919467, WO9923211, WO9929876, WO9942567, WO 9943793, WO9943794, WO 9946399, WO0029560, WO0060058, WO0060059, WO0060060, WO 0114532, WO0134784, WO 0164852, WO0166712, WO0188107, WO0196537, WO02092797, WO 0210355, WO0231124, WO 2004055178, WO2004113551, WO2005001064, WO2005003311, WO 2005018336, WO2005019443, WO2005066338, WO2006002643, WO2006012899, WO2006012902, WO2006031554, WO 2006063594, WO2006066594, WO2006066596, WO2006136161, WO 2008000825, WO2008088493, WO2008092919, WO2008101894, WO2008/112459, WO2009061380, WO2009061381, WO 2009100102, WO2009140504, WO2009149419, WO 2010/059413, WO 2010088447, WO2010091221, WO2010104675, WO2010115021, WO10115028, WO2010117511, WO 2011076123, WO2011076897, WO2011080352, WO2011080353, WO 2011080354, WO2011082425, WO2011082429, WO 2011087836, WO2011098531, WO2013063460, WO2013184577, WO 2014099523, WO2014164777, and WO2015077126. Exemplary commercial amylases include, but are not limited to AMPLIFY®, DURAMYL®, TERMAMYL®, FUNGAMYL®, STAINZYME® STAINZYME PLUS®, STAINZYME PLUS®, STAINZYME ULTRA® EVITY®, and BAN™ (Novozymes); EFFECTENZ™ S 1000, POWERASE™, PREFERENZ™ S 100, PREFERENZ™ S 110, EXCELLENZ™ S 2000, RAPIDASE® and MAXAMYL® P (DuPont).

The compositions and methods provided herein additionally comprise a nuclease, such as a DNase or RNase. Exemplary nucleases include, but are not limited to, those described in WO2015181287, WO2015155350, WO2016162556, WO2017162836, WO2017060475 (e.g. SEQ ID NO: 21), WO2018184816, WO2018177936, WO2018177938, WO2018/185269, WO2018185285, WO2018177203, WO2018184817, WO2019084349, WO2019084350, WO2019081721, WO2018076800, WO2018185267, WO2018185280, and WO2018206553. Other nucleases which can be used in combination with the polypeptides having nuclease activity in the compositions and methods provided herein include those described in Nijland R, Hall M J, Burgess J G (2010) Dispersal of Biofilms by Secreted, Matrix Degrading, Bacterial DNase. PLoS ONE 5(12) and Whitchurch, C. B., Tolker-Nielsen, T., Ragas, P. C., Mattick, J. S. (2002) Extracellular DNA required for bacterial biofilm formation. Science 295: 1487.

The compositions and methods provided can additionally comprise a cellulase. An exemplary cellulase can be a chemically or genetically modified mutant. Exemplary cellulases include but are not limited, to those of bacterial or fungal origin, such as, for example, those described in WO2005054475, WO2005056787, U.S. Pat. Nos. 7,449,318, 7,833,773, 4,435,307; EP 0495257; and U.S. Provisional Appl. No. 62/296,678. Exemplary commercial cellulases include, but are not limited to, CELLUCLEAN®, CELLUZYME®, CAREZYME®, ENDOLASE®, RENOZYME®, and CAREZYME® PREMIUM (Novozymes); REVITALENZ™ 100, REVITALENZ™ 200/220, and REVITALENZ® 2000 (DuPont); and KAC-500(B)™ (Kao Corporation). In some embodiments, cellulases are incorporated as portions or fragments of mature wild-type or variant cellulases, wherein a portion of the N-terminus is deleted (see, e.g., U.S. Pat. No. 5,874,276).

In other embodiments, the composition described herein comprises one or more additional biofilm controlling agents, such as alginate oligomers and probiotics. Alginate oligomers for use in such compositions include those, for example, in U.S. Pat. No. 10,624,920. Probiotics for use in the compositions include those disclosed, for example, in WO2020008053, WO2018060475, WO2017157774, and WO2017142743.

In some embodiments, the laundry detergent compositions described herein comprise at least one chelating agent. Suitable chelating agents may include, but are not limited to copper, iron, and/or manganese chelating agents, and mixtures thereof. In some embodiments, the laundry detergent compositions described herein comprises from about 0.1% to about 15% or even from about 3.0% to about 10% chelating agent by weight of composition.

In some still further embodiments, the laundry detergent compositions described herein comprise at least one deposition aid. Suitable deposition aids include, but are not limited to, polyethylene glycol, polypropylene glycol, polycarboxylate, soil release polymers such as polyterephthalic acid, clays such as kaolinite, montmorillonite, attapulgite, illite, bentonite, halloysite, and mixtures thereof.

In some embodiments, the laundry detergent compositions described herein comprise at least one anti-redeposition agent.

In some embodiments, the laundry detergent compositions described herein comprise one or more dye transfer inhibiting agent. Suitable polymeric dye transfer inhibiting agents include, but are not limited to, polyvinylpyrrolidone polymers, polyamine N-oxide polymers, copolymers of N-vinylpyrrolidone and N-vinylimidazole, polyvinyloxazolidones, and polyvinylimidazoles, or mixtures thereof. In some embodiments, the laundry detergent compositions described herein comprise from about 0.0001% to about 10%, from about 0.01% to about 5%, or even from about 0.1% to about 3% dye transfer inhibiting agent by weight of composition.

In some embodiments, the laundry detergent compositions described herein comprise one or more silicates. In some such embodiments, sodium silicates (e.g., sodium disilicate, sodium metasilicate, and crystalline phyllosilicates) find use. In some embodiments, the laundry detergent compositions described herein comprise from about 1% to about 20% or from about 5% to about 15% silicate by weight of the composition.

In yet further embodiments, the laundry detergent compositions described herein comprise one or more dispersant. Suitable water-soluble organic materials include, but are not limited to the homo- or co-polymeric acids or their salts, in which the polycarboxylic acid comprises at least two carboxyl radicals separated from each other by not more than two carbon atoms.

In some embodiments, the laundry detergent compositions described herein comprise one or more bleach, bleach activator, and/or bleach catalyst. In some embodiments, the laundry detergent compositions described herein comprise inorganic and/or organic bleaching compound(s). Inorganic bleaches may include, but are not limited to perhydrate salts (e.g., perborate, percarbonate, perphosphate, persulfate, and persilicate salts). In some embodiments, inorganic perhydrate salts are alkali metal salts. In some embodiments, inorganic perhydrate salts are included as the crystalline solid, without additional protection, although in some other embodiments, the salt is coated. Suitable salts include, for example, those described in EP2100949. Bleach activators are typically organic peracid precursors that enhance the bleaching action in the course of cleaning at temperatures of 60° C. and below. Bleach activators suitable for use herein include compounds which, under perhydrolysis conditions, give aliphatic peroxycarboxylic acids having preferably from about 1 to about 10 carbon atoms, in particular from about 2 to about 4 carbon atoms, and/or optionally substituted perbenzoic acid. Bleach catalysts typically include, for example, manganese triazacyclononane and related complexes, and cobalt, copper, manganese, and iron complexes, as well as those described in U.S. Pat. Nos. 4,246,612, 5,227,084, 4,810,410, WO9906521, and EP2100949.

In some embodiments, the laundry detergent compositions described herein comprise one or more catalytic metal complex. In some embodiments, a metal-containing bleach catalyst finds use. In other embodiments, the metal bleach catalyst comprises a catalyst system comprising a transition metal cation of defined bleach catalytic activity (e.g., copper, iron, titanium, ruthenium, tungsten, molybdenum, or manganese cations), an auxiliary metal cation having little or no bleach catalytic activity (e.g., zinc or aluminum cations), and a sequestrate having defined stability constants for the catalytic and auxiliary metal cations, particularly ethylenediaminetetraacetic acid, ethylenediaminetetra (methylenephosphonic acid) and water-soluble salts thereof are used (See, e.g., U.S. Pat. No. 4,430,243). In some embodiments, the laundry detergent compositions described herein are catalyzed by means of a manganese compound. Such compounds and levels of use are well known in the art (See, e.g., U.S. Pat. No. 5,576,282). In additional embodiments, cobalt bleach catalysts find use in the laundry detergent compositions described herein. Various cobalt bleach catalysts are known in the art (See, e.g., U.S. Pat. Nos. 5,597,936 and 5,595,967) and are readily prepared by known procedures. Some embodiments are directed to a method of cleaning comprising contacting an effective amount of a cleaning composition described herein with an item or surface comprising a soil, stain or biofilm to hydrolyze the soil, stain or biofilm.

In some further embodiments, the laundry detergent compositions described herein comprise one or more enzyme stabilizer. In some embodiments, the enzyme stabilizer is a water-soluble source of calcium and/or magnesium ions. In some embodiments, the enzyme stabilizers include oligosaccharides, polysaccharides, and inorganic divalent metal salts, including alkaline earth metals, such as calcium salts. In some embodiments, the enzymes employed herein are stabilized by the presence of water-soluble sources of zinc (II), calcium (II) and/or magnesium (II) ions in the finished compositions that provide such ions to the enzymes, as well as other metal ions (e.g., barium (II), scandium (II), iron (II), manganese (II), aluminum (III), tin (II), cobalt (II), copper (II), nickel (II), and oxovanadium (IV)). Chlorides and sulfates also find use in some embodiments. Exemplary oligosaccharides and polysaccharides (e.g., dextrins) are described, for example, in WO07145964. In some embodiments, the laundry detergent compositions described herein contain reversible protease inhibitors selected from a boron-containing compound (e.g., borate, 4-formyl phenyl boronic acid, and phenyl-boronic acid derivatives, such as, e.g., are described in WO9641859); a peptide aldehyde (such as, e.g., is described in WO2009118375 and WO2013004636), and combinations thereof.

The cleaning compositions herein are typically formulated such that, during use in aqueous cleaning operations, the wash water will have a pH of from about 3.0 to about 11. Liquid product formulations are typically formulated to have a neat pH from about 5.0 to about 9.0, more preferably from about 7.5 to about 9. Granular laundry products are typically formulated to have a pH from about 8.0 to about 11.0. Techniques for controlling pH at recommended usage levels include the use of buffers, alkalis, acids, etc., and are well known to those skilled in the art.

Suitable high pH cleaning compositions typically have a neat pH of from about 9.0 to about 11.0, or even a neat pH of from 9.5 to 10.5. Such cleaning compositions typically comprise a sufficient amount of a pH modifier, such as sodium hydroxide, monoethanolamine, or hydrochloric acid, to provide such cleaning composition with a neat pH of from about 9.0 to about 11.0. Such compositions typically comprise at least one base-stable enzyme. In some embodiments, the compositions are liquids, while in other embodiments, they are solids.

In one embodiment, the cleaning compositions include those having a pH of from 7.4 to pH 11.5, or pH 7.4 to pH 11.0, or pH 7.5 to pH 11.5, or pH 7.5 to pH 11.0, or pH 7.5 to pH 10.5, or pH 7.5 to pH 10.0, or pH 7.5 to pH 9.5, or pH 7.5 to pH 9.0, or pH 7.5 to pH 8.5, or pH 7.5 to pH 8.0, or pH 7.6 to pH 11.5, or pH 7.6 to pH 11.0, or pH 7.6 to pH 10.5, or pH 8.7 to pH 10.0, or pH 8.0 to pH 11.5, or pH 8.0 to pH 11.0, or pH 8.0 to pH 10.5, or pH 8.0 to pH 10.0.

Concentrations of detergent compositions in typical wash solutions throughout the world vary from less than about 800 ppm of detergent composition (“low detergent concentration geographies”), for example about 667 ppm in Japan, to between about 800 ppm to about 2000 ppm (“medium detergent concentration geographies”), for example about 975 ppm in U.S. and about 1500 ppm in Brazil, to greater than about 2000 ppm (“high detergent concentration geographies”), for example about 4500 ppm to about 5000 ppm in Europe and about 6000 ppm in high suds phosphate builder geographies.

When a parameter is given cither as a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. The scope of the invention is not intended to be limited to the specific values and examples as recited in the specification.

### EXAMPLES

### Example 1. Identification and Production of Nucleases

A search for nuclease enzymes was performed by protein families scanning [ref. https://www.ncbi.nlm.nih.gov/pmc/articles/PMC6324024/]. Coding sequences containing “DUF1524” domains (https://pfam.xfam.org/family/PF07510) or “Endonuclease_NS” domains (https://pfam.xfam.org/family/Endonuclease_NS) were identified and further analyzed. Genes encoding these nucleases were identified from the sources listed in Table 1, and are assigned the SEQ ID NOs shown on Table 1. The N-terminal signal peptides were predicted by SignalP software version 4.0 (Nordahl Petersen et al. (2011) Nature Methods, 8:785-786). Proteins were expressed in either Bacillus subtilis or Trichoderma reesei using standard cloning and expression techniques.

For expression in Bacillus subtilis, commercially synthesized polynucleotides encoding the gene of interest were inserted into the p2JM103BBI plasmid (Vogtentanz, Protein Expr Purif, 55:40-52, 2007). The expression plasmid included an aprE promoter and the synthetic gene encoding the target protein. In some cases, the aprE signal peptide was also included. For sequences expressed without the signal peptide, and for which the first amino acid of the predicted mature protein of CRC16475-WT is not a methionine residue, a start codon ‘GTG’ was added before the gene-encoding polynucleotides. The plasmid was used to transform B. subtilis cells, and the transformed cells were spread on Luria Agar plates supplemented with 5 ppm Chloramphenicol. The colony with correct insertion confirmed by PCR and sequencing was selected and subjected to fermentation using standard methods.

For expression in Trichoderma reesei, commercially synthesized polynucleotides encoding the gene of interest were inserted into the pGX256 expression vector (described in U.S. Published Application 2011/0136197 A1). The plasmids were transformed into a suitable Trichoderma reesei strain using protoplast transformation (Te'o et al., J. Microbiol. Methods 51:393-99, 2002). The transformants were selected and fermented by the methods described in WO 2016/138315. Protein expression by the transformants was confirmed by SDS-PAGE of culture supernatants. Fungal cell cultures were grown in a defined medium. Clarified culture broths were collected after 96 hours by centrifugation.

Proteins were purified by passage over a HiPrep Phenyl FF 16/10 column, by passage over a HiPrep Q FF column, by passage over a HiPrep SP FF column, or by passage over a combination of these columns as needed to achieve purification.

Purification with the HiPrep Phenyl FF 16/10 column was as follows. Crude material from fermentation was concentrated and ammonium sulfate was added to a final concentration of 1 M. The solution was loaded onto a HiPrep Phenyl FF 16/10 column pre-equilibrated with 20 mM sodium acetate (pH 5.0) supplemented with 1 M ammonium sulfate. The target protein was eluted with a gradient of decreasing ammonium sulfate concentration. Fractions containing the protein of interest were pooled and concentrated.

Purification with the HiPrep Q FF column was as follows. The solution containing the protein of interest was concentrated and buffer exchanged into either 20 mM sodium phosphate (pH 7.0) or 20 mM Tris (pH 8.0) and then loaded onto a HiPrep Q FF column pre-equilibrated with either 20 mM sodium phosphate (pH 7.0) or 20 mM Tris (pH 8.0). The target protein was eluted with a gradient of sodium chloride and the active fractions were pooled and concentrated.

Purification with the HiPrep SP FF column was as follows. The solution containing the protein of interest was concentrated and buffer exchanged into 20 mM sodium phosphate (pH 7.0) and then loaded onto a HiPrep SP FF column pre-equilibrated with 20 mM sodium phosphate (pH 7.0). The target protein was eluted with a gradient of sodium chloride and active protein fractions were pooled and concentrated.

TABLE 1
Sequences, names, and sources of genetic sequence information

| Sequence ID | Name | Source of genetic sequence information |
|---|---|---|
| SEQ ID 1 | TreNuc1 | Trichoderma reesei QM6a |
| SEQ ID 2 | TinNuc1 | Tolypocladium inflatum NRRL 8044 |
| SEQ ID 3 | BdeNuc1 | Blastomyces gilchristii SLH14081 |
| SEQ ID 4 | GteNuc1 | Gelasinospora tetrasperma CBS 560.94 |
| SEQ ID 5 | SdyNuc1 | Streptococcus dysgalactiae H46A |
| SEQ ID 6 | TceNuc1 | Thermobifida cellulosilytica DSM 44535 |
| SEQ ID 7 | RcoNuc1 | Rasamsonia composticola |
| SEQ ID 8 | CcrNuc1 | Chaetosartorya cremea |
| SEQ ID 10 | AbiNuc1 | Aspergillus bisporus CBS 707.71 |
| SEQ ID 11 | AclNuc1 | Aspergillus clavatus NRRL 1 from AspGD |
| SEQ ID 12 | MacNuc1 | Metarhizium acridum CQMa 102 |
| SEQ ID 13 | AocNuc1 | Aspergillus ochraceoroseus SRRC1432 |
| SEQ ID 14 | BciNuc1 | Bacillus cibi |

### Example 2. Dispersal of Pseudomonas Fluorescens Biofilm in Simulations of Repeated Laundry Washes

Pseudomonas fluorescens (ATCC strain 700830) biofilm was formed on 96-well round bottom plates (Corning 2797). Briefly, colonies from an LB-agar plate were used to inoculate fresh Tryptic Soy Broth (TSB, Teknova T11550) followed by OD600 adjustment to 0.1-0.2. Then the cell suspension was transferred to a microtiter plate and the plate was incubated in an oxygen chamber statically for 48 h at 28 degrees C. After decanting and washing the plate 5 times with 1×PBS and air-drying, the biofilm buildup in plates was treated with enzyme solution at 20 ppm or at 100 ppm, prepared in a 1:1200 dilution of Tide Original liquid detergent (the solution alone was used as negative control). The SEQ ID NO. 14 at 100 PPM was included for comparison. For each sample, at least eight replicates were performed. Plates were incubated in an iEMS incubator at 26 degrees C. with shaking at 400 rpm for 400 min. Then the treatment solutions were decanted, and the plate was washed 5 times with Milli-Q water and air dried. After treatment, biofilm was stained by crystal violet solution (0.1%). After 5 min, the excess crystal violet was removed, and the plate was washed 5 times and air dried. Finally, the biofilm bound crystal violet was dissolved in 30% acetic acid solution. Biofilm signal was monitored in terms of absorbance at 590 nm using a spectrophotometer.

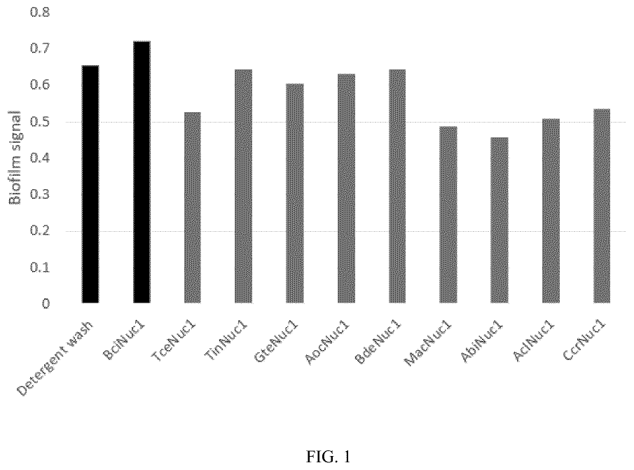

As shown in FIGS. 1 and 2, several nucleases removed more biofilm than the benchmark SEQ ID NO. 14 at 100 PPM enzyme and 20 PPM enzyme dose.

### Example 3. Dispersal of Pseudomonas Fluorescens Biofilm in Simulations of Repeated Laundry Washes

A biofilm dispersal assay was adapted from the procedure described by Pitts et al (2003). Pseudomonas fluorescens (ATCC strain 700830) biofilm was formed in 96-well round bottom plates (Corning 2797). Briefly, colonies from an LB plate were inoculated in fresh TSB media followed by OD600 adjustment to 0.1-0.2. Then the cell suspension was transferred to a microtiter plate and the plate was incubated in oxygen chamber statically for 48 h at 28 degrees C. After washing 5 times with 1×PBS and air-drying, the biofilm buildup in plates was treated with enzyme solution at the concentrations indicated, prepared in a 1:1200 dilution of Tide Original liquid detergent (the solution alone was used as negative control) in water. Enzymes concentrations were 10 ppm, 50 ppm, or 250 ppm as indicated. The SEQ ID NO. 14 was included for comparison. For each sample, eight replicates were performed. Plates were incubated in an iEMS incubator at 26 degrees C. with shaking at 400 rpm for 400 minutes. Then treatment solutions were decanted and the plate was washed 5 times with Milli-Q water and air dried. After treatment, biofilm was stained by crystal violet solution (0.1%). After 5 min, the excess crystal violet was removed and the plate was washed 5 times and air dried. Finally, the biofilm bound crystal violet was dissolved in 30% acetic acid solution. Biofilm was monitored in terms of OD590 nm using a spectrophotometer.

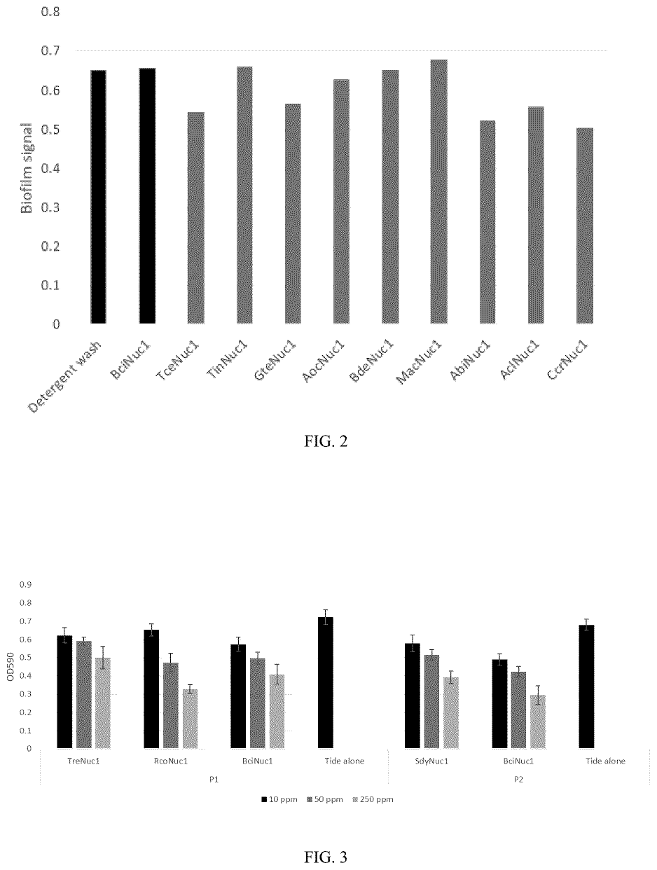

Results are shown in FIG. 3.

### Example 4. Stability in Liquid Laundry Detergent

Nuclease stability in the presence of detergent was determined by preincubating 20 PPM of nuclease in approximately wash concentration detergent, a 1:1200 dilution of liquid Tide Original detergent in water, in a Master Cycler (Eppendorf) at 40° C. for 45 min and then assaying for remaining activity as described below. Following preincubation in detergent, detergent-enzyme solutions were diluted by an amount depending on the level of enzymatic activity of each enzyme in the absence of detergent preincubation. To determine the degree of dilution, activity was initially assayed at 10 PPM, 1 PPM, and 0.1 PPM without detergent preincubation and the lowest concentration that yielded full degradation of the DNA substrate was selected for the assay.

The activity assay was as follows. Enzyme solution was added to an assay solution containing 180 ng of 1.8 kb PCR DNA as target in 1×NEB buffer (containing 50 mM Tris-HCl, 100 mM NaCl, 10 mM MgCl2 and 1 mM DTT, pH 7.9). In a parallel test, non-preincubated samples were tested for their activity both in the presence and absence of Tide dilutions. After incubation in the Master Cycler at 37° C. for 1 hr, the reaction was stopped by adding EDTA to a final concentration of 30 mM and heating at 99° C. for 10 min. Before loading the reaction solution onto a gel, 2.8 kb DNA was added into each well as internal control for following band quantification. The reaction solution was treated with a DNA Clean & Concentrator kit to remove the detergent effect prior to loading on 1% agarose gel with 0.5 μg/ml ethidium bromide on a standard electrophoresis system. The visualized gel band was quantified with the Gel Doc EZ imager with Image Lab software (BIO-RAD). Nuclease residual activity (%) was measured by quantifying the residual target DNA and comparing the signal with that of no-enzyme DNA sample. Complete loss of the DNA band corresponds to 100% residual activity and no loss of the DNA band corresponds to 0% residual activity.

As seen in Table 2, all but one of the nucleases have increased activity relative to SEQ ID NO. 14 after preincubation in wash-concentration detergent.

TABLE 2
Remaining nuclease activity after preincubation in
wash-concentration Tide Original liquid detergent.

| Nuclease | Activity after detergent preincubation |
|---|---|
| BciNuc1 | 14% |
| RcoNuc1 | 0% |
| TreNuc1 | 62% |
| CcrNuc1 | 100% |
| SdyNuc1 | 25% |
| TceNuc1 | 100% |
| TinNuc1 | 100% |
| BdeNuc1 | 100% |
| GteNuc1 | 100% |
| AbiNuc1 | 100% |
| AclNuc1 | 100% |
| AocNuc1 | 100% |
| MacNuc1 | 100% |

### Example 5: Pseudomonas Fluorescens Biofilm Dispersal by Combination of Lysozyme SmaLys1 and Nuclease CcrNuc1

A biofilm dispersal assay was adapted from the procedure described by Pitts et al (Pitts, B., Hamilton, M. A., Zelver, N., Stewart, P. S. (2003) A microtiter-plate screening method for biofilm disinfection and removal. Journal of Microbiological Methods 54: 269-276), briefly as follows. Pseudomonas fluorescens (ATCC strain 700830) biofilm was formed on 96-well round bottom plates (Corning 2797). Briefly, colonies from an LB growth plate were used to inoculate fresh TSB media followed by OD600 adjustment to 0.1-0.2. Then the cell suspension was transferred to a microtiter plate and the plate was incubated in oxygen chamber statically for 48 h at 28° C. After washing 5 times with 1×PBS and air-drying, the biofilm buildup in plates was treated with enzyme solution, prepared in 1:1200 dilution of Tide liquid detergent dilution, a roughly wash-concentration detergent solution. The Tide dilution alone was used as a negative control. For each sample, eight replicates were performed. Plates were incubated in an iEMS incubator at 26° C. with shaking at 400 rpm for 6 hours, simulating multiple washes. Then the treatment solutions were decanted, and the plate was washed 5 times with Milli-Q water and air dried. After treatment, biofilm was stained by crystal violet solution (0.1%). After 5 min, the excess crystal violet was removed, and the plate was washed 5 times and air dried. Finally, the biofilm bound crystal violet was dissolved in 30% acetic acid solution. The residual biofilm was monitored with a spectrophotometer at 590 nm.

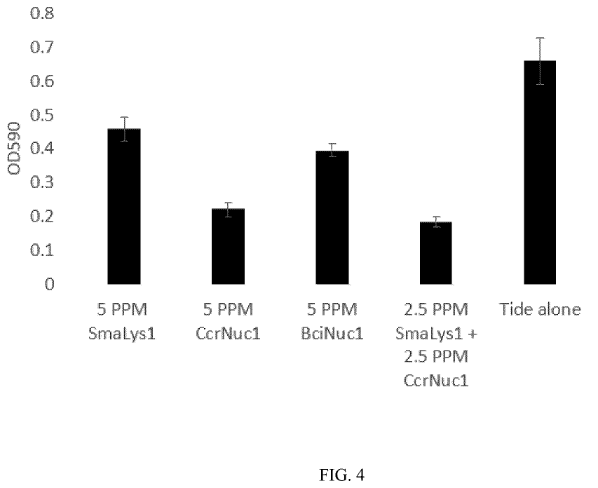

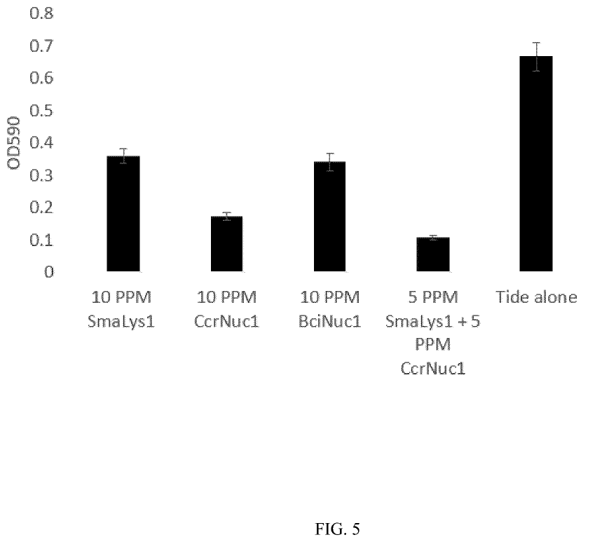

FIGS. 4 and 5 indicate that the combination of lysozyme SmaLys1 and nuclease CcrNuc1 (at the ratio of 1:1) showed improved activity over each single component alone at the same total protein amount, indicating that these two molecules have synergistic effect on biofilm dispersal. Both nuclease CcrNuc1 alone and the combination outperformed SEQ ID NO. 14 at the same protein level.

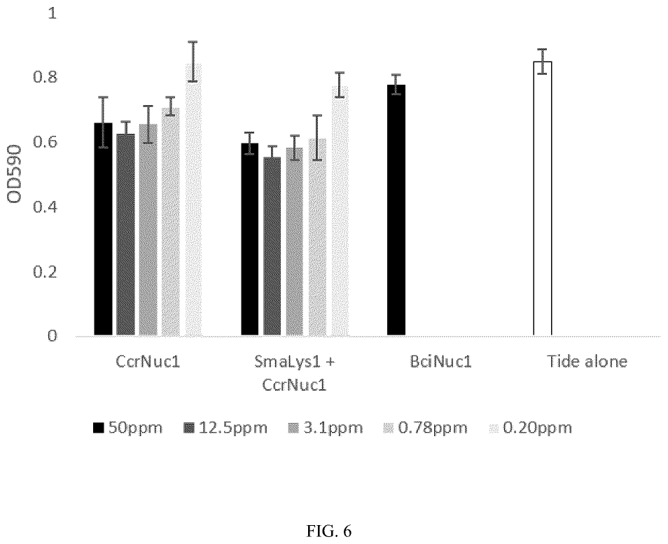

In FIG. 6, the increased performance of combination of lysozyme SmaLys1 and nuclease CcrNuc1 (at the ratio of 1:1) over single component CcrNuc1 was further improved by doing dose response curve. The combination kept working and showed benefit even when the dose decreased to as low as 0.2 ppm.

What is claimed is:

1. A method for preventing, reducing or removing a biofilm comprising contacting the biofilm with a cleaning composition comprising a polypeptide having nuclease activity, wherein the polypeptide having the nuclease activity is a polypeptide having at least 80% sequence identity with an enzyme selected from the group consisting of SEQ ID NO: 1 to 13.

2. The method of claim 1, wherein the biofilm is on a textile or hard surface.

3. The method of claim 2, wherein the hard surface is selected from the group consisting of a laundry machine surface, a dish surface, or a dishwasher surface.

4. The method of any of the preceding claims, wherein the cleaning composition comprises a polypeptide having nuclease activity in an amount selected from 0.001 to 10,000 mg/L, or 0.001 to 2000 mg/L, or 0.01 to 5000 mg/L, or 0.01 to 2000 mg/L, or 0.01 to 1300 mg/L, or 0.1 to 5000 mg/L, or 0.1 to 2000 mg/L, or 0.1 to 1300 mg/L, or 1 to 5000 mg/L, or 1 to 1300 mg/L, or 1 to 500 mg/L, or 10 to 5000 mg/L, or 10 to 1300 mg/L, or 10 to 500 mg/L.

5. The method of any of the preceding claims, wherein the cleaning composition is a laundry composition.

6. A method for preventing, reducing or removing a biofilm from a textile or hard surface comprising: (i) contacting a textile or surface with a polypeptide having nuclease activity or a composition comprising a polypeptide having nuclease activity; and (ii) optionally, rinsing the textile or surface, wherein the polypeptide having the nuclease activity is a polypeptide having at least 80% sequence identity selected from the group consisting of SEQ ID NO: 1 to 13.

7. The method of claim 6, wherein the textile comprises a biofilm on a surface of the textile.

8. The method of claim 7, wherein the biofilm is reduced or removed from the textile.

9. The method of any of the preceding claims, wherein the biofilm is reduced or removed from the article in an amount selected from the groups consisting of at least 5%, 10%, 15%, 20%, 25%, 30%, 35%, 40%, 45%, 50%, 55%, 60%, 65%, 70%, 75%, 80%, 85%, 90%, 95% or greater compared to the amount of the biofilm present on the textile prior to contacting the textile with the polypeptide having nuclease activity or a composition comprising a polypeptide having nuclease activity.

10. The method of any of the preceding claims, wherein the biofilm is measured by staining with crystal violet.

11. The method of any of the preceding claims, wherein the contacting step comprises a polypeptide having nuclease activity in an amount selected from the group consisting of 0.002 to 10,000 mg of protein, 0.005 to 5000 mg of protein, 0.01 to 5000 mg of protein, 0.05 to 5000 mg of protein, 0.05 to 1300 mg of protein, 0.1 to 1300 mg of protein, 0.1 to 500 mg of protein, 0.1 to 100 mg of protein, per liter of wash liquor, or in the amount of at least 0.002 ppm active nuclease.

12. The method of any of the preceding claims, wherein the cleaning composition has a pH of from 7.4 to pH 11.5, or pH 7.4 to pH 11.0, or pH 7.5 to pH 11.5.

13. The method of any of the preceding claims, wherein the polypeptide having nuclease activity is a polypeptide having at least 80%, 85%, 90%, 95%, 98%, 99%, or 100% sequence identity to SEQ ID NO: 8.

14. The method of any of the preceding claims, wherein the contacting step occurs in a wash liquor.

15. The method of any of the preceding claims, wherein the contacting step takes place for an amount of time selected from the group consisting of about 5 minutes to about 10 days, about 5 minutes to about 400 minutes, between about 5 minutes to about 300 minutes, between about 5 minutes to about 250 minutes, between about 5 minutes to about 200 minutes, between about 5 minutes to about 150 minutes, between about 5 minutes to about 100 minutes, between about 5 minutes to about 50 minutes, between about 5 minutes to about 30 minutes.

16. The method of any of the preceding claims, wherein the contacting step takes place at a temperature selected from the group consisting of about 10° to 60° C., between 15° to about 55° C., between 20° to about 50° C. and between 20° to about 45° C.

17. The method of any of the preceding claims, wherein the composition comprising a polypeptide having nuclease activity further comprises a surfactant.

18. The method of claim 17, wherein the surfactant is selected from the group consisting of a non-ionic, ampholytic, semi-polar, anionic, cationic, zwitterionic, and combinations and mixtures thereof.

19. The method of any of the preceding claims, wherein the composition is a detergent composition.

20. The method of any of the preceding claims, wherein the contacting step further includes contacting the textile with one or more additional enzymes selected from the group consisting of acyl transferases, alpha-amylases, beta-amylases, alpha-galactosidases, arabinosidases, aryl esterases, beta-galactosidases, carrageenases, catalases, cellobiohydrolases, cellulases, chondroitinases, cutinases, endo-beta-1,4-glucanases, endo-beta-mannanases, esterases, exo-mannanases, galactanases, glucoamylases, hemicellulases, hyaluronidases, keratinases, laccases, lactases, ligninases, lipases, lipoxygenases, lysozymes, mannanases, metalloproteases, oxidases, oxidoreductases, pectate lyases, pectin acetyl esterases, pectinases, pentosanases, perhydrolases, peroxidases, phenoloxidases, phosphatases, phospholipases, phytases, polygalacturonases, polyesterases, proteases, pullulanases, reductases, rhamnogalacturonases, beta-glucanases, tannases, transglutaminases, xylan acetyl-esterases, xylanases, xyloglucanases, xylosidases, and any combination or mixture thereof.

21. The method of any of the preceding claims, wherein the contacting step takes place in a washing machine or a dishwasher.

22. A detergent composition comprising: (i) a polypeptide having nuclease activity; (ii) a polypeptide having protease activity; (iii) at least one additional polypeptide, wherein the at least one additional polypeptide is an enzyme selected from: DNaseacyl transferases, alpha-amylases, beta-amylases, alpha-galactosidases, arabinosidases, aryl esterases, beta-galactosidases, carrageenases, catalases, cellobiohydrolases, cellulases, chondroitinases, cutinases, endo-beta-1,4-glucanases, endo-beta-mannanases, esterases, exo-mannanases, galactanases, glucoamylases, hemicellulases, hyaluronidases, keratinases, laccases, lactases, ligninases, lipases, lipoxygenases, lysozymes, mannanases, metalloproteases, nucleases, oxidases, oxidoreductases, pectate lyases, pectin acetyl esterases, pectinases, pentosanases, perhydrolases, peroxidases, phenoloxidases, phosphatases, phospholipases, phytases, polygalacturonases, polyesterases, proteases, pullulanases, reductases, rhamnogalacturonases, beta-glucanases, tannases, transglutaminases, xylan acetyl-esterases, xylanases, xyloglucanases, xylosidases, and any combination or mixture thereof; and (iv) a surfactant, wherein the polypeptide having the nuclease activity is a polypeptide having at least 80% sequence identity selected from the group consisting of SEQ ID NO: 1 to 13.

23. The composition of claim 22, wherein the surfactant is selected from the group consisting of a non-ionic. ampholytic, semi-polar, anionic, cationic, zwitterionic, and combinations and mixtures thereof.

24. The composition of claim 22, wherein the composition comprises between about 0.1% to about 60%, about 1% to about 50%, or about 5% to about 40% surfactant by weight of the composition.

25. The composition of claim 22, wherein the composition further comprises one or more adjunct materials selected from the group consisting of builders, bleaches, bleach activators, bleach catalysts, other enzymes, enzyme stabilizing systems, chelants, optical brighteners, soil release polymers, dye transfer agents, dispersants, suds suppressors, dyes, perfumes, colorants, filler salts, hydrotropes, photoactivators, fluorescers, fabric conditioners, hydrolyzable surfactants, preservatives, anti-oxidants, anti-shrinkage agents, anti-wrinkle agents, germicides, fungicides, color speckles, silvercare, anti-tarnish and/or anti-corrosion agents, alkalinity sources, solubilizing agents, carriers, processing aids, pigments, and pH control agents.

26. The composition of claim 22, wherein the nuclease is a DNase.

27. A method for reducing malodor associated with a textile or hard surface comprising: (i) contacting a textile or hard surface with a polypeptide having nuclease activity or a composition comprising a polypeptide having nuclease activity; and (ii) optionally, rinsing the textile or surface, wherein the polypeptide having the nuclease activity is a polypeptide having at least 80% sequence identity selected from a group consisting of SEQ ID NO: 1 to 13.

28. The method of claim 27, wherein the textile comprises a biofilm on a surface of the textile or hard surface.

29. The method of claim 28, wherein the biofilm is reduced or removed from the textile.

30. The method of any of claims 27 to 28, wherein the malodor is reduced at least 5%, 10%, 15%, 20%, 25%, 30%, 35%, 40%, 45%, 50%, 55%, 60%, 65%, 70%, 75%, 80%, 85%, 90%, 95% or greater compared to the amount of the malodor present prior to contacting the textile or hard surface with the polypeptide having nuclease activity or a composition comprising a polypeptide having nuclease activity.

31. The method of any of claims 27 to 30, wherein the contacting step comprises the use a polypeptide having nuclease activity in an amount selected from the group consisting of 0.002 to 10,000 mg of protein, 0.005 to 5000 mg of protein. 0.01 to 5000 mg of protein, 0.05 to 5000 mg of protein, 0.05 to 1300 mg of protein, 0.1 to 1300 mg of protein, 0.1 to 500 mg of protein, 0.1 to 100 mg of protein, per liter of wash liquor, or in the amount of at least 0.002 ppm active nuclease.

32. The method of any of claims 27-31, wherein the polypeptide having nuclease activity is a polypeptide having at least 85%, 90%, 95%, 96%, 97%, 98%, 99%, or 100% sequence identity selected from a group consisting of SEQ ID NO: 1 to 13.

33. The method of any of claims 27-32, wherein the contacting step occurs in a wash liquor.

34. The method of any of claims 27-33, wherein the contacting step takes place at a temperature selected from the group consisting of about 10° to 60° C., between 15° to about 55° C., between 20° to about 50° C. and between 20° to about 45° C.

35. The method of any of claims 27-34, wherein the composition comprising a polypeptide having nuclease activity further comprises a surfactant.

36. The method of claim 35, wherein the surfactant is selected from the group consisting of a non-ionic, ampholytic, semi-polar, anionic, cationic, zwitterionic, and combinations and mixtures thereof.

37. The method of any of claims 27-36, wherein the composition is a detergent composition.

38. The method of any of claims 27-37, wherein the contacting step further includes contacting the textile with one or more additional enzymes selected from the group consisting of acyl transferases, alpha-amylases, beta-amylases, alpha-galactosidases, arabinosidases, aryl esterases, beta-galactosidases, carrageenases, catalases, cellobiohydrolases, cellulases, chondroitinases, cutinases, endo-beta-1,4-glucanases, endo-beta-mannanases, esterases, exo-mannanases, galactanases, glucoamylases, hemicellulases, hyaluronidases, keratinases, laccases, lactases, ligninases, lipases, lipoxygenases, lysozymes, mannanases, metalloproteases, oxidases, oxidoreductases, pectate lyases, pectin acetyl esterases, pectinases, pentosanases, perhydrolases, peroxidases, phenoloxidases, phosphatases, phospholipases, phytases, polygalacturonases, polyesterases, proteases, pullulanases, reductases, rhamnogalacturonases, beta-glucanases, tannases, transglutaminases, xylan acetyl-esterases, xylanases, xyloglucanases, xylosidases, and any combination or mixture thereof.

39. The method of any of claims 27-38, wherein the contacting step takes place in a washing machine or a dishwasher.

40. An isolated polypeptide or active fragment thereof having nuclease activity, wherein the polypeptide comprises an amino acid sequence having at least 75%, 80%, 85%, 90%, 91%, 92%, 93%, 94%, 95%, 96%, 97%, 98%, 99%, or 100% sequence identity with an amino acid sequence selected from the group consisting of SEQ ID NO: 1 to 13.

41. A method for preventing, reducing or removing a biofilm comprising contacting the biofilm with a cleaning composition comprising a polypeptide having nuclease activity and a lysozyme.

42. A detergent composition comprising: (i) a polypeptide having nuclease activity and (ii) a lysozyme.
